SEQUENCE LISTING (134 sequences)

SEQ ID NO: 1 - protein, 115 aa, Artificial Sequence
  artificial antibody variable domain
AQVLTQTASSVSAAVGGTVTISCQSSESVYNNNRLSWFQQKPGQPPKQLIYSASSLASGVPSRFKGSGSGTQFTLTISDLECDDAATYYCQGEFSCSSADCFTFGGGTEVVVKGD

SEQ ID NO: 2 - protein, 120 aa, Artificial Sequence
  artificial antibody variable domain
QSVEESGGRLVTPGTPLTLTCTVSGFPLSSYAMIWVRQAPGKGLEWIGMILRAGNIYYASWAKGRFTISKTSTTVDLKITSPTTEDTATYFCARRQYNTDGYPIGIGDLWGPGTLVTVSS

SEQ ID NO: 3 - protein, 115 aa, Artificial Sequence
  artificial antibody variable domain
AQVLTQTPSSVSAVVGGTVTISCQSSESVYSNNRLSWFQQKPGQPPKLLIYSASTLASGVPSRFKGSGSGTQFTLTITDLECDDAATYFCQGEFSCSSVDCFSFGGGTEVVVKGD

SEQ ID NO: 4 - protein, 120 aa, Artificial Sequence
  artificial antibody variable domain
QSLEESGGRLVTPGTPLTLTCTVSGFPLSAYAMIWVRQAPGKGLEWIGMIIRSGTVYYANWAKGRFTISKTSTTVDLKITSPTTEDTATYFCARRHYNADGYPIGIGDLWGPGTLVTVSS

SEQ ID NO: 5 - protein, 115 aa, Artificial Sequence
  artificial antibody variable domain
AQVLTQTPSSVSAAVGGTVTISCQSNENIYSNNRLSWFQQKPGQPPNQLIYSASSLASGVPSRFKGSGSGTQFTLTISDLECDDAATYYCQGEFNCNSADCFTFGGGTEVVVKGD

SEQ ID NO: 6 - protein, 120 aa, Artificial Sequence
  artificial antibody variable domain
QSLEESGGRLVTPGTPLTLTCTVSGFPLNRYAMLWVRQAPGKGLEWIGLITRADKKYYASWAKGRFTISKTSTTVDLEITGPTTEDTATYFCARRHYNTDGYPIAIGDLWGPGTLVTVSS

SEQ ID NO: 7 - protein, 115 aa, Artificial Sequence
  artificial antibody variable domain
AQVLTQTPSSVSAAVGGTVTINCQSSQSVYNNNRLSWFQQKPGQPPKLLIYTTSSLASGVPSRFKGSGSGTEFTLTISDLECADAATYYCQGEFSCSRADCFNFGGGTEVVVKGD

SEQ ID NO: 8 - protein, 120 aa, Artificial Sequence
  artificial antibody variable domain
QSLEESGGRLVKPDETLTLTCTVSGFPLSSYAMGWFRQAPGKGLEWIGMILRSDNTYYASWAKGRFTISKTSTTVDLKITSPTTEDTATYFCARRHYNASGNPIAIGDLWGPGTLVTVSS

SEQ ID NO: 9 - protein, 115 aa, Artificial Sequence
  artificial antibody variable domain
AQVLTQTPSSVSAAVGGTVTISCQSSESVYNNKRLSWFQQKPGQPPKQLIYTASSLASGVPSRFKGSGSGTQFTLTISDLECDDAATYYCQGEFTCSNADCFTFGGGTEVVVKGD

SEQ ID NO: 10 - protein, 120 aa, Artificial Sequence
  artificial antibody variable domain
QSVEESGGRLVTPGTPLTLTCTVSGFPLSSYAMIWVRQAPGKGLEWIGMILRAGNIYYASWVKGRVTISKTSTTVDLKITSPTTEDTATYFCARRHYNREGYPIGIGDLWGPGTLVTVSS

SEQ ID NO: 11 - protein, 115 aa, Artificial Sequence; identical to SEQ ID NO: 5
  artificial antibody variable domain

SEQ ID NO: 12 - protein, 120 aa, Artificial Sequence
  artificial antibody variable domain
QSLEESGGRLVTPGTPLTLTCTVSGFPLNRYAMLWVRQAPGKGLEWIGLITRADKKYYASWAKGRFTISKTSTTVDLEITGPTTEDTATYFCARRHYNTDGYPVAIGDLWGPGTLVTVSS

SEQ ID NO: 13 - protein, 115 aa, Artificial Sequence
  artificial antibody variable domain
AQVLTQTPSSVSAAVGGTATISCQSNENIYSNNRLSWFQQKAGQPPNQLIYSASSLASGVPSRFKGSGSGTQFTLTISDLECDDAATYYCQGEFSCSSADCFTFGGGTEVVVKGD

SEQ ID NO: 14 - protein, 120 aa, Artificial Sequence
  artificial antibody variable domain
QSLEESGGRLVTPGTPLTLTCTVSGFPLSSFAMLWVRQAPGKGLEWIGMIMRAHNMYYASWAKGRFTISKTSTTVDLEITSPTTEDTATYFCARRHYNTYGYPIAIGDLWGPGTLVTVSS

SEQ ID NO: 15 - protein, 115 aa, Artificial Sequence
  artificial antibody variable domain
AQVLTQTPSSVSAAVGGTVTINCQSSQSVYNNNRLSWFQQKPGQPPKLLIYTASSLASGVPSRFKGSGSGTEFTLTISDLECADAATYYCQGEFSCSSADCFTFGGGTEVVVKGD

SEQ ID NO: 16 - protein, 120 aa, Artificial Sequence
  artificial antibody variable domain
QSLEESGGRLVTPGTPLTLTCTVSGFPLSSYAMGWFRQAPGKGLEWIGMILRADNTYYASWVNGRFTISKTSTTVDLKITSPTTEDTATYFCARRHYNTYGYPVAIGDLWGPGTLVTVSS

SEQ ID NO: 17 - protein, 115 aa, Artificial Sequence
  artificial antibody variable domain
AQVLTQTPSSVSATVGGTVTISCQSNENIYSNNRLSWFQQKPGQPPKLLIYSASSLASGVPSRFKGSGSGTQFTLTISDLECDDAATYYCQGEFNCNSADCFTFGGGTEVVVKGD

SEQ ID NO: 18 - protein, 120 aa, Artificial Sequence
  artificial antibody variable domain
QSLEESGGRLVTPGTPLTLTCTVSGFPLSRYAMLWVRQAPGKGLEWIGLITRADNKYYASWAKGRFTISKTSTTVDLEITSPTTEDTATYFCARRHYNTDGYPIAIGDLWGPGTLVTVSS

SEQ ID NO: 19 - protein, 115 aa, Artificial Sequence
  artificial antibody variable domain
AQVLTQTXSSVSAXVGGTXTIXCQSXXXXYXNXRLSWFQQKXGQPPXXLIYXXSXLASGVPSRFKGSGSGTXFTLTIXDLECXDAATYXCQGEFXCXXXDCFXFGGGTEVVVKGD

SEQ ID NO: 20 - protein, 120 aa, Artificial Sequence
  artificial antibody variable domain
QSXEESGGRLVXPXXXLTLTCTVSGFPLXXXAMXWXRQAPGKGLEWIGXIXRXXXXYYAXWXXGRXTISKTSTTVDLXITXPTTEDTATYFCARRXYNXXGXPXXIGDLWGPGTLVTVSS

SEQ ID NO: 21 - protein, 114 aa, Artificial Sequence
  artificial antibody variable domain
DIQMTQSPSSLSASVGDRVTITCQSSESVYNNKRLSWYQQKPGKAPKLLIYTASSLASGVPSRFSGSGSGTDFTLTISSLQPEDFATYYCQGEFTCSNADCFTFGQGTKLTVLG

SEQ ID NO: 22 - protein, 123 aa, Artificial Sequence
  artificial antibody variable domain
EVQLVESGGGLVQPGGSLRLSCAASGFPLSSYAMIWVRQAPGKGLEWIGMILRAGNIYYASWVKGRFTISRDNSKNTVYLQMNSLRAEDTAVYYCARRHYNREGYPIGIGDLWGQGTLVTVSS

SEQ ID NO: 23 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 24 - protein, 95 aa, Cynomolgus
DGNEEMGSITQTPYQVSISGTTVILTCSQHLGSEAQWQHNGKNKEDSGDRLFLPEFSEMEQSGYYVCYPRGSNPEDASHHLYLKARVCENCMEMD

SEQ ID NO: 25 - protein, 15 aa, Homo sapiens
DGNEEMGGITQTPYK

SEQ ID NO: 26 - protein, 15 aa, Homo sapiens
GNEEMGGITQTPYKV

SEQ ID NO: 27 - protein, 15 aa, Homo sapiens
NEEMGGITQTPYKVS

SEQ ID NO: 28 - protein, 15 aa, Homo sapiens
EEMGGITQTPYKVSI

SEQ ID NO: 29 - protein, 15 aa, Homo sapiens
EMGGITQTPYKVSIS

SEQ ID NO: 30 - protein, 15 aa, Homo sapiens
MGGITQTPYKVSISG

SEQ ID NO: 31 - protein, 15 aa, Homo sapiens
GGITQTPYKVSISGT

SEQ ID NO: 32 - protein, 15 aa, Homo sapiens
GITQTPYKVSISGTT

SEQ ID NO: 33 - protein, 15 aa, Homo sapiens
ITQTPYKVSISGTTV

SEQ ID NO: 34 - protein, 15 aa, Homo sapiens
TQTPYKVSISGTTVI

SEQ ID NO: 35 - protein, 15 aa, Artificial Sequence
  artificial peptide
DGNEEMGGITAAPYK

SEQ ID NO: 36 - protein, 15 aa, Artificial Sequence
  artificial peptide
GNEEMGGITQAAYKV

SEQ ID NO: 37 - protein, 15 aa, Artificial Sequence
  artificial peptide
NEEMGGITQTAAKVS

SEQ ID NO: 38 - protein, 15 aa, Artificial Sequence
  artificial peptide
EEMGGITQTPAAVSI

SEQ ID NO: 39 - protein, 15 aa, Artificial Sequence
  artificial peptide
EMGGITQTPYAASIS

SEQ ID NO: 40 - protein, 15 aa, Artificial Sequence
  artificial peptide
MGGITQTPYKAAISG

SEQ ID NO: 41 - protein, 15 aa, Artificial Sequence
  artificial peptide
GGITQTPYKVAASGT

SEQ ID NO: 42 - protein, 15 aa, Artificial Sequence
  artificial peptide
GITQTPYKVSAAGTT

SEQ ID NO: 43 - protein, 15 aa, Artificial Sequence
  artificial peptide
ITQTPYKVSIAATTV

SEQ ID NO: 44 - protein, 15 aa, Artificial Sequence
  artificial peptide
TQTPYKVSISAATVI

SEQ ID NO: 45 - protein, 16 aa, Artificial Sequence
  artificial peptide
EDGNEEMGGITQTPYK

SEQ ID NO: 46 - protein, 16 aa, Artificial Sequence
  artificial peptide
EEEMGGITQTPYKVSI

SEQ ID NO: 47 - protein, 16 aa, Artificial Sequence
  artificial peptide
EGGITQTPYKVSISGT

SEQ ID NO: 48 - protein, 16 aa, Artificial Sequence
  artificial peptide
ETQTPYKVSISGTTVI

SEQ ID NO: 49 - protein, 16 aa, Artificial Sequence
  artificial peptide
EPYKVSISGTTVILTC

SEQ ID NO: 50 - protein, 16 aa, Artificial Sequence
  artificial peptide
EVSISGTTVILTCPQY

SEQ ID NO: 51 - protein, 16 aa, Artificial Sequence
  artificial peptide
ESGTTVILTCPQYPGS

SEQ ID NO: 52 - protein, 16 aa, Artificial Sequence
  artificial peptide
ETVILTCPQYPGSEIL

SEQ ID NO: 53 - protein, 16 aa, Artificial Sequence
  artificial peptide
ELTCPQYPGSEILWQH

SEQ ID NO: 54 - protein, 16 aa, Artificial Sequence
  artificial peptide
EPQYPGSEILWQHNDK

SEQ ID NO: 55 - protein, 16 aa, Artificial Sequence
  artificial peptide
KDGNEEMGGITQTPYK

SEQ ID NO: 56 - protein, 16 aa, Artificial Sequence
  artificial peptide
KEEMGGITQTPYKVSI

SEQ ID NO: 57 - protein, 16 aa, Artificial Sequence
  artificial peptide
KGGITQTPYKVSISGT

SEQ ID NO: 58 - protein, 16 aa, Artificial Sequence
  artificial peptide
KTQTPYKVSISGTTVI

SEQ ID NO: 59 - protein, 16 aa, Artificial Sequence
  artificial peptide
KPYKVSISGTTVILTC

SEQ ID NO: 60 - protein, 16 aa, Artificial Sequence
  artificial peptide
KVSISGTTVILTCPQY

SEQ ID NO: 61 - protein, 16 aa, Artificial Sequence
  artificial peptide
KSGTTVILTCPQYPGS

SEQ ID NO: 62 - protein, 16 aa, Artificial Sequence
  artificial peptide
KTVILTCPQYPGSEIL

SEQ ID NO: 63 - protein, 16 aa, Artificial Sequence
  artificial peptide
KLTCPQYPGSEILWQH

SEQ ID NO: 64 - protein, 16 aa, Artificial Sequence
  artificial peptide
KPQYPGSEILWQHNDK

SEQ ID NO: 65 - protein, 17 aa, Artificial Sequence
  artificial peptide
CDGNEEMGGITQTPYKC

SEQ ID NO: 66 - protein, 17 aa, Artificial Sequence
  artificial peptide
CGNEEMGGITQTPYKVC

SEQ ID NO: 67 - protein, 17 aa, Artificial Sequence
  artificial peptide
CNEEMGGITQTPYKVSC

SEQ ID NO: 68 - protein, 17 aa, Artificial Sequence
  artificial peptide
CEEMGGITQTPYKVSIC

SEQ ID NO: 69 - protein, 17 aa, Artificial Sequence
  artificial peptide
CEMGGITQTPYKVSISC

SEQ ID NO: 70 - protein, 17 aa, Artificial Sequence
  artificial peptide
CMGGITQTPYKVSISGC

SEQ ID NO: 71 - protein, 17 aa, Artificial Sequence
  artificial peptide
CGGITQTPYKVSISGTC

SEQ ID NO: 72 - protein, 17 aa, Artificial Sequence
  artificial peptide
CGITQTPYKVSISGTTC

SEQ ID NO: 73 - protein, 17 aa, Artificial Sequence
  artificial peptide
CITQTPYKVSISGTTVC

SEQ ID NO: 74 - protein, 17 aa, Artificial Sequence
  artificial peptide
CTQTPYKVSISGTTVIC

SEQ ID NO: 75 - protein, 22 aa, Artificial Sequence
  artificial peptide
CDGNEEMGGITQTPYKVSISGC

SEQ ID NO: 76 - protein, 22 aa, Artificial Sequence
  artificial peptide
CNEEMGGITQTPYKVSISGTTC

SEQ ID NO: 77 - protein, 22 aa, Artificial Sequence
  artificial peptide
CEMGGITQTPYKVSISGTTVIC

SEQ ID NO: 78 - protein, 22 aa, Artificial Sequence
  artificial peptide
CGGITQTPYKVSISGTTVILTC

SEQ ID NO: 79 - protein, 22 aa, Artificial Sequence
  artificial peptide
CITQTPYKVSISGTTVILTSPC

SEQ ID NO: 80 - protein, 22 aa, Artificial Sequence
  artificial peptide
CQTPYKVSISGTTVILTSPQYC

SEQ ID NO: 81 - protein, 22 aa, Artificial Sequence
  artificial peptide
CPYKVSISGTTVILTSPQYPGC

SEQ ID NO: 82 - protein, 22 aa, Artificial Sequence
  artificial peptide
CKVSISGTTVILTSPQYPGSEC

SEQ ID NO: 83 - protein, 22 aa, Artificial Sequence
  artificial peptide
CSISGTTVILTSPQYPGSEILC

SEQ ID NO: 84 - protein, 22 aa, Artificial Sequence
  artificial peptide
CSGTTVILTSPQYPGSEILWQC

SEQ ID NO: 85 - protein, 29 aa, Artificial Sequence
  artificial peptide
CDGNEEMGGITQTPCDGNEEMGGITQTPC

SEQ ID NO: 86 - protein, 29 aa, Artificial Sequence
  artificial peptide
CDGNEEMGSITQTPCDGNEEMGSITQTPC

SEQ ID NO: 87 - protein, 29 aa, Artificial Sequence
  artificial peptide
CEEMGGITQTPYKVCDGNEEMGGITQTPC

SEQ ID NO: 88 - protein, 29 aa, Artificial Sequence
  artificial peptide
CEEMGSITQTPYQVCDGNEEMGSITQTPC

SEQ ID NO: 89 - protein, 29 aa, Artificial Sequence
  artificial peptide
CGGITQTPYKVSISCDGNEEMGGITQTPC

SEQ ID NO: 90 - protein, 29 aa, Artificial Sequence
  artificial peptide
CGSITQTPYQVSISCDGNEEMGSITQTPC

SEQ ID NO: 91 - protein, 29 aa, Artificial Sequence
  artificial peptide
CTQTPYKVSISGTTCDGNEEMGGITQTPC

SEQ ID NO: 92 - protein, 29 aa, Artificial Sequence
  artificial peptide
CTQTPYQVSISGTTCDGNEEMGSITQTPC

SEQ ID NO: 93 - protein, 29 aa, Artificial Sequence
  artificial peptide
CPYKVSISGTTVILCDGNEEMGGITQTPC

SEQ ID NO: 94 - protein, 29 aa, Artificial Sequence
  artificial peptide
CPYQVSISGTTVILCDGNEEMGSITQTPC

SEQ ID NO: 95 - protein, 15 aa, Artificial Sequence
  artificial peptide
AGNEEMGGITQTPYK

SEQ ID NO: 96 - protein, 15 aa, Artificial Sequence
  artificial peptide
DGNEEMGGITQTPYK

SEQ ID NO: 97 - protein, 15 aa, Artificial Sequence
  artificial peptide
GGNEEMGGITQTPYK

SEQ ID NO: 98 - protein, 15 aa, Artificial Sequence
  artificial peptide
HGNEEMGGITQTPYK

SEQ ID NO: 99 - protein, 15 aa, Artificial Sequence
  artificial peptide
LGNEEMGGITQTPYK

SEQ ID NO: 100 - protein, 15 aa, Artificial Sequence
  artificial peptide
MGNEEMGGITQTPYK

SEQ ID NO: 101 - protein, 15 aa, Artificial Sequence
  artificial peptide
NGNEEMGGITQTPYK

SEQ ID NO: 102 - protein, 15 aa, Artificial Sequence
  artificial peptide
PGNEEMGGITQTPYK

SEQ ID NO: 103 - protein, 15 aa, Artificial Sequence
  artificial peptide
QGNEEMGGITQTPYK

SEQ ID NO: 104 - protein, 15 aa, Artificial Sequence
  artificial peptide
RGNEEMGGITQTPYK

SEQ ID NO: 105 - protein, 15 aa, Artificial Sequence
  artificial peptide
AGNEEMGSITQTPYQ

SEQ ID NO: 106 - protein, 15 aa, Artificial Sequence
  artificial peptide
DGNEEMGSITQTPYQ

SEQ ID NO: 107 - protein, 15 aa, Artificial Sequence
  artificial peptide
GGNEEMGSITQTPYQ

SEQ ID NO: 108 - protein, 15 aa, Artificial Sequence
  artificial peptide
HGNEEMGSITQTPYQ

SEQ ID NO: 109 - protein, 15 aa, Artificial Sequence
  artificial peptide
LGNEEMGSITQTPYQ

SEQ ID NO: 110 - protein, 15 aa, Artificial Sequence
  artificial peptide
MGNEEMGSITQTPYQ

SEQ ID NO: 111 - protein, 15 aa, Artificial Sequence
  artificial peptide
NGNEEMGSITQTPYQ

SEQ ID NO: 112 - protein, 15 aa, Artificial Sequence
  artificial peptide
PGNEEMGSITQTPYQ

SEQ ID NO: 113 - protein, 15 aa, Artificial Sequence
  artificial peptide
QGNEEMGSITQTPYQ

SEQ ID NO: 114 - protein, 15 aa, Artificial Sequence
  artificial peptide
RGNEEMGSITQTPYQ

SEQ ID NO: 115 - protein, 15 aa, Artificial Sequence
  artificial peptide
AEMGGITQTPYKVSI

SEQ ID NO: 116 - protein, 15 aa, Artificial Sequence
  artificial peptide
DEMGGITQTPYKVSI

SEQ ID NO: 117 - protein, 15 aa, Artificial Sequence
  artificial peptide
GEMGGITQTPYKVSI

SEQ ID NO: 118 - protein, 15 aa, Artificial Sequence
  artificial peptide
HEMGGITQTPYKVSI

SEQ ID NO: 119 - protein, 15 aa, Artificial Sequence
  artificial peptide
LEMGGITQTPYKVSI

SEQ ID NO: 120 - protein, 15 aa, Artificial Sequence
  artificial peptide
MEMGGITQTPYKVSI

SEQ ID NO: 121 - protein, 15 aa, Artificial Sequence
  artificial peptide
NEMGGITQTPYKVSI

SEQ ID NO: 122 - protein, 15 aa, Artificial Sequence
  artificial peptide
PEMGGITQTPYKVSI

SEQ ID NO: 123 - protein, 15 aa, Artificial Sequence
  artificial peptide
QEMGGITQTPYKVSI

SEQ ID NO: 124 - protein, 15 aa, Artificial Sequence
  artificial peptide
REMGGITQTPYKVSI

SEQ ID NO: 125 - protein, 15 aa, Artificial Sequence
  artificial peptide
AEMGSITQTPYQVSI

SEQ ID NO: 126 - protein, 15 aa, Artificial Sequence
  artificial peptide
DEMGSITQTPYQVSI

SEQ ID NO: 127 - protein, 15 aa, Artificial Sequence
  artificial peptide
GEMGSITQTPYQVSI

SEQ ID NO: 128 - protein, 15 aa, Artificial Sequence
  artificial peptide
HEMGSITQTPYQVSI

SEQ ID NO: 129 - protein, 15 aa, Artificial Sequence
  artificial peptide
LEMGSITQTPYQVSI

SEQ ID NO: 130 - protein, 15 aa, Artificial Sequence
  artificial peptide
MEMGSITQTPYQVSI

SEQ ID NO: 131 - protein, 15 aa, Artificial Sequence
  artificial peptide
NEMGSITQTPYQVSI

SEQ ID NO: 132 - protein, 15 aa, Artificial Sequence
  artificial peptide
PEMGSITQTPYQVSI

SEQ ID NO: 133 - protein, 15 aa, Artificial Sequence
  artificial peptide
QEMGSITQTPYQVSI

SEQ ID NO: 134 - protein, 15 aa, Artificial Sequence
  artificial peptide
REMGSITQTPYQVSI

(19) United States
(12) Patent Application Publication
(10) Pub. No.: US 2016/0090416 A1
(43) Pub. Date: Mar. 31, 2016

(54) NOVEL ANTIBODIES

(71) Applicant: NUMAB AG, Waedenswil (CH)

(72) Inventors: Tea GUNDE, Zuerich (CH); Sebastian MEYER, Eggenwil (CH); David URECH, Jona (CH)

(73) Assignee: NUMAB AG, Waedenswil (CH)

(21) Appl. No.: 14/893,497

(22) PCT Filed: May 28, 2014
(86) PCT No.: PCT/EP2014/001460

§ 371 Date: Nov. 23, 2015

(30) Foreign Application Priority Data
May 28, 2013 (EP) 13002769.1
Oct. 25, 2013 (EP) 13005113.9
May 12, 2014 (EP) PCT/EP2014/001282

(51) Int. Cl. C07K 16/28

(52) U.S. Cl. CPC C07K 16/2809; C07K 2317/33; C07K 2317/92; C07K 2317/24; C07K 2317/34; C07K 2317/75

(57) ABSTRACT

The present invention relates to novel antibodies, which combine high affinity with high potency, particularly novel antibodies against a novel epitope.

### FIELD OF THE INVENTION

The present invention relates to novel antibodies, which combine high affinity with high potency, particularly novel antibodies against a novel epitope.

### BACKGROUND OF THE INVENTION

This invention relates to novel anti-CD3 antibodies, which combine high affinity with high potency, and in particular novel antibodies, which specifically recognize a novel CD3 epitope.

The T cell receptor or TCR is a molecule found on the surface of T lymphocytes (or T cells) that is responsible for recognizing antigens bound to major histocompatibility complex (MHC) molecules on the surface of antigen presenting cells (APC). The binding between TCR and antigen is of relatively low affinity. When the TCR engages with antigen and MHC, the T lymphocyte is activated through a series of biochemical events mediated by associated enzymes, co-receptors, specialized accessory molecules, and activated or released transcription factors.

The TCR is associated with other molecules like CD3, which possess three distinct chains (γ, δ, and ε) in mammals, and either a ζ2 (CD247) complex or a ζ/η complex. These accessory molecules have transmembrane regions and are vital to propagating the signal from the TCR into the cell; the cytoplasmic tail of the TCR is extremely short, making it unlikely to participate in signaling. The CD3- and ζ-chains, together with the TCR, form what is known as the T cell receptor complex.

CD3ε is a type I transmembrane protein expressed on the surface of certain T cells. It participates in the T cell receptor (TCR) complex and interacts with other domains of this complex. One of these interaction partners is CD3γ, which binds to CD3ε in a 1:1 stoichiometry (De la Hera et al, J. Exp. Med. 1991; 173: 7-17). FIG. 5 shows a schematic view of the TCR complex, including CD3ε/CD3γ. It is believed that binding of the TCR to the MHC-peptide complex on the surface of an antigen presenting cell (APC) and subsequent movement of the T cell along the APC leads to a certain rotation of the TCR complex resulting in a dislocation of CD3ε and CD3γ relative to each other, which is required for efficient TCR signaling and therefore activation of T-cells. Certain antibodies against CD3ε have been demonstrated to induce TCR signaling while others did not. TCR-activating antibodies typically bind to an exposed epitope on CD3ε (see FIG. 5, “agonistic epitope”), whereas some non-stimulatory antibodies have been demonstrated to bind to the interface between CD3ε and CD3γ or to concomitantly bind to CD3ε and CD3γ (see FIG. 5, “antagonistic epitope”), thus possibly interfering with the relative displacement of CD3ε and CD3γ (Kim et al, JBC.2009; 284: 31028-31037).

It is well established that peptide-MHC complexes bind TCR with low affinity and fast off rate (Matsui et al, Science.1991; 254: 1788-1791; Weber et al, Nature. 1992; 356: 793-796). It has been suggested that this low affinity is instrumental to allow a few peptide-MHC complexes to serially trigger many TCRs (Valitutti et al, Nature. 1995; 375: 148-151) by repeated binding and dissociation. This serial triggering is critical to sustain signaling over time, allowing T cells to eventually reach the activation threshold (Valitutti et al, Immunol. Today. 1997; 18: 299-304; Lanzavecchia et al, Cell. 1999; 96: 1-4). This notion is supported by the finding that, when compared to peptide-MHC complexes, high-affinity anti-CD3 antibodies do not efficiently stimulate T cells, since they trigger TCR with a 1:1 stoichiometry (Viola et al, Science 1996; 273: 104-106), suggesting that low-affinity antibodies may be more effective in stimulating T cells via TCR signaling because of their ability to repeatedly dissociate and re-bind to CD3ε. Indeed, in a direct comparison of three derivatives of the anti-CD3ε antibody TR66, which all bind with different affinities, wild-type TR66 having an intermediate affinity showed best efficacy in T cell activation when compared to its derivatives that have either higher or lower affinities (Bortoletto et al, J. Immuno. 2002; 32:3102-3107). Thus, a K_{D} at around that of TR66 is ideal for the stimulation of T cells. The affinity of TR66 has been determined by use of surface-plasmon resonance (SPR) technology as well as by flow-cytometry, yielding equilibrium dissociation constants of 2.6×10⁻⁷ M (Moore et al, Blood. 2011; 117: 4542-4551) and 1.0×10⁻⁷ M (Amann et al, Cancer Res. 2008; 68: 143-151), respectively. In line with this, it has been recommended to use anti-CD3 antibodies with an affinity of less than 10⁻⁸ M (U.S. Pat. No. 7,112,324), and the T cell-stimulatory antibodies that have been published for human therapeutic use, bind with affinities to human CD3ε in the same range. Therefore, according to the theory of serial TCR triggering and in agreement with published results for anti-CD3ε antibodies, monoclonal antibodies with affinities significantly better than the ones published are not expected to be more potent stimulators of T cells but in contrast are expected to be weaker activators.

Some of the published antibodies against CD3ε have been generated via immunization of animals with T cell preparations and subsequent isolation of monoclonal antibodies by the so-called hybridoma procedure. The weakness of this approach is that the unselective immune response against various antigens of foreign (human) T cells in the animal, on one hand, and the poor efficiency of the hybridoma procedure on the other hand, decrease the probability to identify monoclonal antibodies with T cell-stimulatory activity, also because these agonistic antibodies may represent a minority in the entirety of anti-CD3ε antibodies. Immunization with a linear peptide spanning the targeted epitope increases the selectivity of the immune response, may, however, result in antibodies that do not recognize the native full-length CD3ε or that may exert non-optimal TCR stimulation.

For the immunization of animals with other type-I transmembrane proteins it has been particularly useful to use the purified extracellular domain (ECD). However, purified ECD of CD3ε tends to aggregate, and aggregates may have an altered structure as compared to the native protein. Further this approach may preferentially lead to antibodies binding to the interface between CD3ε and CD3γ. In contrast, the complex of CD3ε and CD3γ produced as a single-chain protein, connected by a flexible peptide linker, can be purified in a monomeric fraction and in its native conformation (Kim et al, JMB. 2000; 302: 899-916). Immunization of animals with such a CD3ε/γ single-chain protein may however lead to antibodies concomitantly binding to CD3ε and CD3γ, which would result in antagonistic effects.

Several antibodies directed against human CD3ε have been developed in the past.

Monoclonal antibody SP34 is a murine antibody that cross-reacts with non-human primate CD3, and that is also capable of inducing cell proliferation on both human and non-human primate PBMCs (Pessano et al., The T3/T cell receptor complex: antigenic distinction between the two 20-kD T3 (T3δ and T3ε) subunits. EMBO J 4 (1985) 337-344).

WO 2007/042261 and WO 2008/119567, both assigned to Micromet, disclose cross-reactive binders directed against the epitopes FSEXE and QDGNE, respectively, in CD3ε. In opposition proceedings filed by several opponents against granted European patent EP 2 155 783 (based on the regional phase of WO 2008/119567), it is submitted that SP34 is binding to epitope QDGNE as well.

However, despite the fact that many attempts have been made to address the issue of obtaining anti-CD3 antibodies, or to binding molecules in general, with particularly advantageous properties, so far these attempts have had limited success.

Thus, there remained still a large unmet need to develop novel CD3 binding molecules, in particular novel anti-CD3 antibodies, for high affinity, which is not limiting for high potency. Additionally, there is still a large unmet need to develop novel CD3 binding molecules, in particular novel anti-CD3 antibodies, for high affinity, which are cross-reactive with other species, in particular with non-human primates such as cynomolgus monkeys.

The solution for this problem that has been provided by the present invention, i.e. CD3-binding molecules, in particular anti-CD3 antibodies obtained by genetic immunization of rabbits and screening of affinity matured memory B-cells, and in particular CD3-binding molecules, in particular anti-CD3 antibodies, with specificity for a novel agonistic epitope, has so far not been achieved or suggested by the prior art.

### SUMMARY OF THE INVENTION

The present invention relates to novel isolated CD3-binding molecules, in particular isolated antibodies or functional fragments thereof, each comprising a binding region, particularly an antigen-binding region, wherein said binding molecules, in particular said antibodies or functional fragments thereof, are specific for an epitope of human CD3, particularly for a novel agonistic epitope of CD3ε, wherein said binding molecules, in particular said isolated antibodies or functional fragments thereof, have a higher affinity than the prior art antibodies, particularly OKT-3 and/or TR66, while simultaneously exhibiting a higher potency.

Thus, in a first aspect, the present invention relates to an isolated binding molecule comprising a binding region that is specific for an epitope of human CD3ε, in particular to an isolated antibody or functional fragment thereof comprising an antigen-binding region, wherein said epitope comprises amino acid residue N4 as residue that is critical for binding.

In a second aspect, the present invention relates to a novel isolated CD3-binding molecule that is specific for an epitope of human CD3, wherein said isolated CD3-binding molecule is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M, in particular to an isolated antibody or functional fragment thereof comprising an antigen-binding region that is specific for an epitope of human CD3, wherein said antibody or functional fragment thereof, is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

In a third aspect, the present invention relates to an isolated antibody or functional fragment thereof comprising an antigen-binding region that is specific for an epitope of human CD3, wherein said antibody or functional fragment thereof, when tested in an IgG format, upon cross-linking, is inducing T-cell activation at least 1.5-fold stronger than antibodies OKT-3 or TR66 after 24 h of stimulation at an IgG concentration of 1.25 μg/ml.

In a fourth aspect, the present invention relates to an isolated antibody or functional fragment thereof comprising an antigen-binding region that is specific for an epitope of human CD3, wherein said antibody or functional fragment thereof, when tested in an IgG format upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

In a fifth aspect, the present invention relates to an isolated antibody or functional fragment thereof comprising an antigen-binding region that is specific for an epitope of human CD3, wherein said antibody or functional fragment thereof, when tested in an IgG format, upon cross-linking, is resulting in a dose-dependent homogeneous activation state of T-cells.

In a sixth aspect, the present invention relates to an isolated antibody or functional fragment thereof comprising an antigen-binding region that is specific for an epitope of human CD3, wherein said antibody or functional fragment thereof, when tested in an IgG format, (i) is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M; and (iia), upon cross-linking, is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; (iic) is resulting in a dose-dependent homogeneous activation state of T-cells; and/or (iid) is specific for an epitope of human CD3ε, wherein said epitope comprises amino acid residue N4 as residue that is critical for binding.

In a seventh aspect, the present invention relates to an isolated binding molecule, particularly an isolated antibody or functional fragment thereof, binding to essentially the same epitope as the isolated antibody or functional fragment thereof of Sections [0078] to [0080], [0083] to [0085] and [0089].

In an eighth aspect, the present invention relates to a pharmaceutical composition comprising a binding molecule of the present invention, in particular an isolated antibody or functional fragment thereof, and optionally a pharmaceutically acceptable carrier and/or excipient.

In a ninth aspect, the present invention relates to a nucleic acid sequence or a collection of nucleic acid sequences encoding a binding molecule of the present invention, in particular an isolated antibody or functional fragment thereof.

In a tenth aspect, the present invention relates to a vector or a collection of vectors comprising the nucleic acid sequence or a collection of nucleic acid sequences of the present invention.

In an eleventh aspect, the present invention relates to a host cell, particularly an expression host cell, comprising the nucleic acid sequence or the collection of nucleic acid sequences of the present invention, or the vector or collection of vectors of the present invention.

In a twelfth aspect, the present invention relates to a method for producing a binding molecule of the present invention, in particular an isolated antibody or functional fragment thereof, comprising the step of expressing the nucleic acid sequence or the collection of nucleic acid sequences of the present invention, or the vector or collection of vectors of the present invention, or the host cell, particularly an expression host cell, of the present invention.

In a thirteenth aspect, the present invention relates to a method for generating an isolated antibody or functional antibody fragment in accordance with the present invention comprising the steps of:
a) Immunization of rabbits with a CD3ε-expressing plasmid to present the native full-length CD3ε on the surface of host cells;
b) Clonal isolation of affinity matured memory B-cells that interact with the CD3ε/γ single-chain using fluorescence activated cell-sorting;
c) Cultivation of single sorted B cells in a co-cultivation system that does not require immortalization of sorted clones;
d) Screening of B cell culture supernatants in a cell-based ELISA to identify antibodies binding to the native CD3ε embedded in the TCR complex on the surface of T cells.

In a fourteenth aspect the present invention relates to a particular epitope of human CD3 epsilon comprising exclusively amino acid residues of CD3 epsilon that are not located in the interface between CD3 epsilon and CD3 gamma and that still can be bound by an antibody in the context of the native TCR expressed on T cells, binding of which by a cross-linked antibody of the invention is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; and/or (iic) is resulting in a dose-dependent homogeneous activation state of T-cells.

In a fifteenth aspect, the present invention relates to a method for identifying a binding molecule comprising a binding region that is specific for a novel epitope of human CD3ε, comprising the step of (a) selecting from one or more molecules binding to human CD3 at least one binding molecule, which comprises a binding region that is specific for an epitope of human CD3ε, wherein said epitope comprises amino acid residue N4 as residue that is critical for binding.

### BRIEF DESCRIPTION OF THE DRAWINGS

FIG. 1 shows the phylogenetic clustering of joined VH and VL CDR Sequences from monoclonal rabbit antibodies.

FIG. 2 shows binding of purified monoclonal rabbit antibodies to Jurkat T cells.

FIG. 3 shows the stimulation of CD69 expression by cross-linked anti-CD3ε mAbs. The potential of purified monoclonal rabbit anti-CD3 antibodies and comparator antibodies TR66 and OKT-3 to induce T-cell activation was assessed by measurement of CD69 expression. Three different concentrations of cross-linked antibodies were used to stimulate Jurkat cells and CD69 expression was assessed by flow-cytometry 24 h later. Antibody concentrations were 1.25 μg/ml (a), 5.0 μg/ml (b) and 20 μg/ml (c).

FIG. 4 shows the stimulation of CD69 by cross-linked rabbit mAbs over time. The potential of purified monoclonal rabbit anti-CD3 antibodies to induce T-cell activation was assessed by measurement of CD69 expression. Cross-linked antibodies were used at a concentration of 5.0 μg/ml to stimulate Jurkat cells and CD69 expression was assessed by flow-cytometry 0, 4, 15, 24, 48 and 72 h later. For the qualitative detection of CD69 expression the mean fluorescence intensity (MFI), reflecting the signal intensity at the geometric mean, was measured for both, the negative control as well as for the test antibodies. The difference of the MFI between test antibody and negative control (ΔMFI) was calculated as a measure for CD69 expression.

FIG. 5 shows a simplified schematic view of the TCR complex, including CD3ε/CD3γ.

FIG. 6 shows the results of epitope mapping experiments for prior art antibodies: (a) epitope mapping of antibody SP34 (see file history of EP 2 155 788); (b) epitope mapping of Micromet antibody (see EP 2 155 788/WO 2008/119567; FIG. 6 shows the results of binding experiments of single alanine mutants, where a decrease of binding for a given mutant indicates the relevance of the corresponding wild-type amino acid residue for antibody binding (i.e. low bar=highly relevant for binding).

FIG. 7 shows the results of epitope mapping experiments by ELISA for antibodies of the present invention (clone-02, clone-03, clone-06); FIG. 7 shows the results of binding experiments in a peptide scan analysis. 15mer linear arrays derived from human CD3ε, residues 1-15 in which each position is substituted by 18 amino acids (all natural amino acids except cysteine) were probed with 0.1 μg/ml of each antibody to study amino acid specificities affecting binding to the epitope. Decrease in binding signals in ELISA is given, (a) for each substitution individually, and (b) averaged over the 18 different substitutions for each position. The height of a bar in FIG. 7b indicates the relevance of the corresponding wild-type amino acid residue for antibody binding (i.e. large bar=highly relevant for binding).

FIG. 8 shows binding of anti-CD3×anti-IL5R scDbs to Jurkat T-cells and CHO-IL5R cells. Binding of A) Construct 1, B) Construct 2 and C) Construct 3 to Jurkat T-cells and CD3-negative Jurkat cells and binding of D) Construct 1, E) Construct 2 and F) Construct 3 to IL5R-CHO cells as well as wild-type CHO cells was assessed by flow cytometry. Construct 1, Construct 2 and Construct 3 have the same anti-IL5R moiety but 3 different anti-CD3 moieties that bind to CD3 with diverse affinities (1.15×10⁻⁸ M for Construct 1, 2.96×10⁻⁸ M for Construct 2, and 1.23×10⁻⁷ M for Construct 3); Construct 1=comprises the humanized variable domain of clone-06; Construct 2=comprises the humanized variable domain of clone-02; Construct 3=comprises the humanized variable domain of clone-03.

FIG. 9 shows the specific stimulation of interleukin-2 secretion by cross-linking of cytotoxic T-cells with target cells by scDbs. CD8+ T-cells were incubated with increasing concentrations of scDbs in presence of CHO-IL5R or CHO cells. Interleukin-2 concentrations in culture supernatants were measured by ELISA after 16 hours of incubation; Construct 1=comprises the humanized variable domain of clone-06; Construct 2=comprises the humanized variable domain of clone-02; Construct 3=comprises the humanized variable domain of clone-03.

FIG. 10 shows the specific lysis of human IL5R-expressing CHO cells by anti-CD3×anti-IL5R scDbs. CD8+ T-cells were incubated with increasing concentrations of scDbs in presence of CHO-IL5R or CHO cells. Target cells (CHO-IL5R and CHO) were labeled with cell tox green dye and cell lysis was determined by measurement of fluorescence intensity after 88 hours of incubation; Construct 1=comprises the humanized variable domain of clone-06; Construct 2=comprises the humanized variable domain of clone-02; Construct 3=comprises the humanized variable domain of clone-03.

### DETAILED DESCRIPTION OF THE INVENTION

The peculiarity of this invention compared to former anti-CD3 antibodies is the fact that the novel isolated antibodies or functional fragments thereof comprising antigen-binding regions that are specific for an epitope of human CD3 have higher affinities than the prior art antibodies, particularly OKT-3 and/or TR66, while simultaneously exhibiting higher potencies.

Thus, in a first aspect, the present invention relates to an isolated binding molecule comprising a binding region that is specific for an epitope of human CD3ε, in particular to an isolated antibody or functional fragment thereof comprising an antigen-binding region, wherein said epitope comprises amino acid residue N4 as residue that is critical for binding.

In the context of the present invention, an amino acid residue is to be considered “critical for binding”, when the binding affinity of a binding molecule to a peptide comprising said amino acid residue position is reduced to at least 50%, particularly to at least 25%, more particularly to at least 10%, and most particularly to at least 5% of the binding affinity to the wild-type peptide sequence, when said critical amino acid residue is exchanged by alanine. and/or when the average signal intensity resulting from binding to a peptide comprising said amino acid residue position as determined by the ELISA of Example 7 is reduced to at least 50%, particularly to at least 25%, and most particularly to at least 10% of the binding signal to the wild-type peptide sequence, when said critical amino acid residue is separately exchanged by each of the other natural amino acid residues except cysteine.

In particular embodiments, said epitope further comprises amino acid residue E6 as residue that is involved in binding. In particular embodiments, said epitope further comprises amino acid residue E6 as residue that is critical for binding.

In the context of the present invention, an amino acid residue is to be considered “involved in binding”, when the binding affinity of a binding molecule is reduced to at least 80%, when said amino acid residue is exchanged by alanine, and/or when the average signal intensity resulting from binding to a peptide comprising said amino acid residue position as determined by the ELISA of Example 7 is reduced to at least 80%, when said amino acid residue is separately exchanged by each of the other natural amino acid residues except cysteine.

In particular embodiments, said binding molecule is an antibody or functional fragment thereof.

In particular embodiments, said binding molecule, particularly said isolated antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In particular embodiments, said binding molecule, in particular said antibody or functional fragment thereof, is binding to human CD3 with an equilibrium dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

In particular embodiments, said binding molecule is an antibody or functional fragment thereof, which, when tested in an IgG format, upon cross-linking, is inducing T-cell activation at least 1.5-fold stronger than antibodies OKT-3 or TR66 after 24 h of stimulation at an IgG concentration of 1.25 μg/ml.

In particular embodiments, said binding molecule is an antibody or functional fragment thereof, which, when tested in an IgG format upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

In particular embodiments, said binding molecule is an antibody or functional fragment thereof, which, when tested in an IgG format, upon cross-linking, is resulting in a dose-dependent activation state of T-cells that is less heterogeneous when compared to activation by OKT-3 or TR66.

In a second aspect, the present invention relates to a novel isolated CD3-binding molecule that is specific for an epitope of human CD3, wherein said isolated CD3-binding molecule is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M, in particular to an isolated antibody or functional fragment thereof comprising an antigen-binding region that is specific for an epitope of human CD3, wherein said antibody or functional fragment thereof, is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

In particular embodiments, said binding molecule, particularly said isolated antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In a third aspect, the present invention relates to an isolated antibody or functional fragment thereof comprising an antigen-binding region that is specific for an epitope of human CD3, wherein said antibody or functional fragment thereof, when tested in an IgG format, upon cross-linking, is inducing T-cell activation at least 1.5-fold stronger than antibodies OKT-3 or TR66 after 24 h of stimulation at an IgG concentration of 1.25 μg/ml.

In particular embodiments, said binding molecule, particularly said isolated antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In a fourth aspect, the present invention relates to an isolated antibody or functional fragment thereof comprising an antigen-binding region that is specific for an epitope of human CD3, wherein said antibody or functional fragment thereof, when tested in an IgG format, upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

In particular embodiments, said binding molecule, particularly said isolated antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In a fifth aspect, the present invention relates to an isolated antibody or functional fragment thereof comprising an antigen-binding region that is specific for an epitope of human CD3, wherein said antibody or functional fragment thereof, when tested in an IgG format, upon cross-linking, is resulting in a dose-dependent activation state of T-cells that is less heterogeneous when compared to activation by OKT-3 or TR66.

In particular embodiments, said binding molecule, particularly said isolated antibody or functional fragment thereof, is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In a sixth aspect, the present invention relates to an isolated antibody or functional fragment thereof comprising an antigen-binding region that is specific for an epitope of human CD3, wherein said antibody or functional fragment thereof, when tested in an IgG format, (i) is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M; and (iia), upon cross-linking, is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; (iic) is resulting in a dose-dependent activation state of T-cells that is less heterogeneous when compared to activation by OKT-3 or TR66; and/or (iid) is specific for an epitope of human CD3ε, wherein said epitope comprises amino acid residue N4 as residue that is critical for binding. For the sake of clarity, according to this embodiment, the isolated antibody or functional fragment thereof has the property of (i) and additionally at least one of the properties according to (iia) to (iid).

In particular such embodiments, said isolated antibody or functional fragment thereof, is additionally cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In the context of the present invention, the term “antibody” is used as a synonym for “immunoglobulin” (Ig), which is defined as a protein belonging to the class IgG, IgM, IgB, IgA, or IgD (or any subclass thereof), and includes all conventionally known antibodies and functional fragments thereof. A “functional fragment” of an antibody/immunoglobulin is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and Ito 113 of VH; numbering according to WO 97/08320). In the case of rabbit antibodies, the CDR regions are indicated in Table 4 (see below). A preferred class of immunoglobulins for use in the present invention is IgG. “Functional fragments” of the invention include the domain of a F(ab′)2 fragment, a Fab fragment and scFv. The F(ab′)2 or Fab may be engineered to minimize or completely remove the intermolecular disulphide interactions that occur between the CH1 and CL domains.

As used herein, a binding molecule is “specific to/for”, “specifically recognizes”, or “specifically binds to” a target, such as human CD3 (or an epitope of human CD3), when such binding molecule is able to discriminate between such target biomolecule and one or more reference molecule(s), since binding specificity is not an absolute, but a relative property. In its most general form (and when no defined reference is mentioned), “specific binding” is referring to the ability of the binding molecule to discriminate between the target biomolecule of interest and an unrelated biomolecule, as determined, for example, in accordance with a specificity assay methods known in the art. Such methods comprise, but are not limited to Western blots, ELISA, RIA, ECL, IRMA tests and peptide scans. For example, a standard ELISA assay can be carried out. The scoring may be carried out by standard colour development (e.g. secondary antibody with horseradish peroxide and tetramethyl benzidine with hydrogen peroxide). The reaction in certain wells is scored by the optical density, for example, at 450 nm. Typical background (=negative reaction) may be about 0.1 OD; typical positive reaction may be about 1 OD. This means the ratio between a positive and a negative score can be 10-fold or higher. Typically, determination of binding specificity is performed by using not a single reference biomolecule, but a set of about three to five unrelated biomolecules, such as milk powder, BSA, transferrin or the like.

In the context of the present invention, the term “about” or “approximately” means between 90% and 110% of a given value or range.

However, “specific binding” also may refer to the ability of a binding molecule to discriminate between the target biomolecule and one or more closely related biomolecule(s), which are used as reference points. Additionally, “specific binding” may relate to the ability of a binding molecule to discriminate between different parts of its target antigen, e.g. different domains, regions or epitopes of the target biomolecule, or between one or more key amino acid residues or stretches of amino acid residues of the target biomolecule.

In the context of the present invention, the term “epitope” refers to that part of a given target biomolecule that is required for specific binding between the target biomolecule and a binding molecule. An epitope may be continuous, i.e. formed by adjacent structural elements present in the target biomolecule, or discontinuous, i.e. formed by structural elements that are at different positions in the primary sequence of the target biomolecule, such as in the amino acid sequence of a protein as target, but in close proximity in the three-dimensional structure, which the target biomolecule adopts, such as in the bodily fluid.

In one embodiment, the epitope is located on the epsilon chain of human CD3.

In certain embodiments, said binding to human CD3E is determined by determining the affinity of said antibody or functional fragment thereof in an IgG format to the purified extracellular domain of heterodimeric CD3εγ of human origin using a surface plasmon resonance experiment.

In a particular embodiment, the following conditions are used, as shown in Example 1: MASS-1 SPR instrument (Sierra Sensors); capture antibody: antibody specific for the Fc region of said IgG immobilized on an SPR-2 Affinity Sensor chip, Amine, Sierra Sensors, using a standard amine-coupling procedure; two-fold serial dilutions of human heterodimeric single-chain CD3_{εγ} extracellular domain ranging from 90 to 2.81 nM, injection into the flow cells for 3 min and dissociation of the protein from the IgG captured on the sensor chip for 5 min, surface regeneration after each injection cycle with two injections of 10 mM glycine-HCl, calculation of the apparent dissociation (kd) and association (ka) rate constants and the apparent dissociation equilibrium constant (K_{D}) with the MASS-1 analysis software (Analyzer, Sierra Sensors) using one-to-one Langmuir binding model.

In particular embodiments, said inducing of T-cell activation according to (iia) and/or (iic) is determined by determining the stimulation of CD69 expression by said isolated antibody or functional fragment thereof in an IgG format.

In a particular embodiment, the following conditions are used, as shown in Example 3: stimulation of Jurkat cells (100,000 cells/well) for 24 h with 20 μg/ml, 5 μg/ml and 1.25 μg/ml of said isolated antibody or functional fragment thereof in an IgG format after prior cross-linking by addition of 3-fold excess of an anti-IgG antibody (control: OKT3 (BioLegend, Cat. No. 317302) or TR66 (Novus Biologicals, Cat. No. NBP1-97446), cross-linking with rabbit anti-mouse IgG antibody (JacksonImmuno Research, Cat. No. 315-005-008)); cell staining for CD69 expression after stimulation using a Phycoerithrin (PE)-labeled antibody specific for human CD69 (BioLegend, Cat. No. 310906), analysis with a flow cytometer (FACS aria III, Becton Dickinson); negative control: unstimulated Jurkat cells incubated with the cross-linking antibody stained with said anti-CD69 antibody.

In particular embodiments, said longer lasting T-cell activation according to (iib) is determined by determining the time course of stimulation of CD69 expression by said isolated antibody or functional fragment thereof in an IgG format.

In a particular embodiment, the following conditions are used, as shown in Example 3: stimulation of 100,000 Jurkat cells/well for 0 h, 4 h, 15 h, 24 h, 48 h and 72 h with 5 μg/ml of said isolated antibody or functional fragment thereof in an IgG format anti-CD3 antibodies that have been cross-linked as in [0071] and analysis of CD69 expression by flow cytometry as in [0071].

In particular embodiments, said inducing of T-cell activation according to (iia) and/or (iic) is determined by determining the stimulation of IL-2 secretion by said isolated antibody or functional fragment thereof in an IgG format.

In a particular embodiment, the following conditions are used, as shown in Example 4: stimulation of Jurkat cells (200,000 cells/well) with said isolated antibody or functional fragment thereof in an IgG format at a concentration of 5 μg/ml using 4 different assay setups: (a) stimulation of Jurkat cells with said isolated antibody or functional fragment thereof in an IgG format cross-linked by addition of 3-fold higher concentrations of an anti IgG antibody (control: OKT3 (BioLegend, Cat. No. 317302) or TR66 (Novus Biologicals, Cat. No. NBP1-97446), cross-linking with rabbit anti-mouse IgG antibody (JacksonImmuno Research, Cat. No. 315-005-008)); (b) T-cell activation in absence of cross-linking antibody; (c) immobilization of said cross-linking antibodies on the tissue culture plates by over-night incubation; (d) immobilization of said isolated antibody or functional fragment thereof in an IgG format (or of control antibodies) on the tissue culture plate by over-night incubation in absence of cross-linking antibodies; in each setup, one hour after addition, stimulation of cells with 10 ng/ml PMA and collection of supernatant after 24, 48 and 72 h to measure IL-2 release, quantified using a commercially available ELISA (BioLegend, Cat. No. 431801).

In particular embodiments, the antibody or functional fragment thereof is (i) a rabbit antibody or functional fragment thereof, or (ii) an antibody or functional fragment thereof obtained by humanizing the rabbit antibody or functional fragment thereof of (i).

Methods for the humanization of rabbit antibodies are well known to anyone of ordinary skill in the art (see, for example, Borras et al., J Biol Chem. 2010 Mar. 19; 285(12):9054-66; Rader et al, The FASEB Journal, express article 10.1096/fj.02-0281fje, published online Oct. 18, 2002; Yu et al (2010) A Humanized Anti-VEGF Rabbit Monoclonal Antibody Inhibits Angiogenesis and Blocks Tumor Growth in Xenograft Models. PLoS ONE 5(2): e9072. doi:10.1371/journal.pone.0009072).

In particular embodiments, said isolated antibody or functional fragment thereof comprises an antigen-binding region comprising a VH domain comprising a combination of one CDR1, one CDR2 and one CDR3 region present in SEQ ID NOs: 2, 4, 6, 8, 10, 12, 14, 16, 18, and 20, particularly SEQ ID NOs: 4, 6, and 10, more particularly SEQ ID NO: 10, particularly wherein said VH domain comprises framework domains selected from the framework domains present in SEQ ID NOs: 2, 4, 6, 8, 10, 12, 14, 16, 18, and 20, particularly SEQ ID NOs: 4, 6, and 10, more particularly SEQ ID NO: 10, and a VL domain comprising a combination of one CDR1, one CDR2 and one CDR3 region present in SEQ ID NOs: 1, 3, 5, 7, 9, 11, 13, 15, 17, and 19, particularly SEQ ID NOs: 3, 5, and 9, more particularly SEQ ID NO: 9, particularly wherein said VL domain comprises framework domains selected from the framework domains present in SEQ ID NOs: 1, 3, 5, 7, 9, 11, 13, 15, 17, and 19, particularly SEQ ID NOs: 3, 5, and 9, more particularly SEQ ID NO: 9. In particular embodiments, the VL domain comprises framework domains selected from the framework domains present in SEQ ID NO: 21 and the VH domain comprises framework domains selected from the framework domains present in SEQ ID NO: 22. In other particular embodiments, the VL domain comprises framework domains that are variants of the framework domains present in SEQ ID NO: 21 and/or the VH domain comprises framework domains that are variants of the framework domains present in SEQ ID NO: 22, particularly variants comprising one or more non-human donor amino acid residues, particularly donor amino acid residues present in one of the sequences selected from SEQ ID NOs: 1 to 20, instead of the corresponding human acceptor amino residues present in SEQ ID NO: 21 and/or 22.

In particular embodiments, said isolated antibody or functional fragment thereof comprises an antigen-binding region comprising a VH domain comprising the combination of CDR1, CDR2 and CDR3 present in one of SEQ ID NOs: 2, 4, 6, 8, 10, 12, 14, 16, 18, and 20, particularly SEQ ID NOs: 4, 6, and 10, more particularly SEQ ID NO: 10, particularly wherein said VH domain comprises the combination of framework domains present in one of SEQ ID NOs: 2, 4, 6, 8, 10, 12, 14, 16, 18, and 20, particularly SEQ ID NOs: 4, 6, and 10, more particularly SEQ ID NO: 10, and a VL domain comprising the combination of CDR1, CDR2 and CDR3 present in one of SEQ ID NOs: 1, 3, 5, 7, 9, 11, 13, 15, 17, and 19, particularly SEQ ID NOs: 3, 5, and 9, more particularly SEQ ID NO: 9, particularly wherein said VL domain comprises the combination of framework domains present in one of SEQ ID NOs: 1, 3, 5, 7, 9, 11, 13, 15, 17, and 19, particularly SEQ ID NOs: 3, 5, and 9, more particularly SEQ ID NO: 9. In particular embodiments, the VL domain comprises framework domains selected from the framework domains present in SEQ ID NO: 21 and the VH domain comprises framework domains selected from the framework domains present in SEQ ID NO: 22. In other particular embodiments, the VL domain comprises framework domains that are variants of the framework domains present in SEQ ID NO: 21 and/or the VH domain comprises framework domains that are variants of the framework domains present in SEQ ID NO: 22, particularly variants comprising one or more non-human donor amino acid residues, particularly donor amino acid residues present in one of the sequences selected from SEQ ID NOs: 1 to 20, instead of the corresponding human acceptor amino residues present in SEQ ID NO: 21 and/or 22.

In particular embodiments, said isolated antibody or functional fragment thereof comprises an antigen-binding region comprising a VH domain selected from SEQ ID NOs: 2, 4, 6, 8, 10, 12, 14, 16, 18, and 20, particularly SEQ ID NOs: 4, 6, and 10, more particularly SEQ ID NO: 10, and a VL domain selected from SEQ ID NOs: 1, 3, 5, 7, 9, 11, 13, 15, 17, and 19, particularly SEQ ID NOs: 3, 5, and 9, more particularly SEQ ID NO: 9. In other particular embodiments, the VH domain is a variant of a VH domain selected from SEQ ID NOs: 2, 4, 6, 8, 10, 12, 14, 16, 18, and 20, particularly SEQ ID NOs: 4, 6, and 10, more particularly SEQ ID NO: 10, and/or the VL domain is a variant of a VL domain selected from SEQ ID NOs: 1, 3, 5, 7, 9, 11, 13, 15, 17, and 19, particularly SEQ ID NOs: 3, 5, and 9, more particularly SEQ ID NO: 9, particularly a variant comprising one or more amino acid residue exchanges in the framework domains and/or in CDR residues not involved in antigen binding.

Methods for the identification of amino acid residues in framework regions suitable for exchange, e.g. by homologous amino acid residues, are well known to one of ordinary skill in the art, including, for example, analysis of groups of homologous sequences for the presence of highly conserved residues (which are particularly kept constant) and variegated sequence positions (which may be modified, particularly by one of the residues naturally found at that position).

Methods for the identification of an amino acid residues in the CDR regions suitable for exchange, e.g. by homologous amino acid residues, are well known to one of ordinary skill in the art, including, for example, analysis of structures of antibody binding domains, particularly of structures of antibody binding domains in a complex with antigens for the presence of antigen-interacting residues (which are particularly kept constant) and sequence positions not in contact with the antigen (which may be modified).

In particular other embodiments, said isolated antibody or functional fragment thereof comprises an antigen-binding region comprising a VH domain selected from SEQ ID NOs: 2, 4, 6, 8, 10, 12, 14, 16, 18, 20, and 22, particularly SEQ ID NOs: 4, 6, 10, and 22, more particularly SEQ ID NO: 10, and 22, and a VL domain selected from SEQ ID NOs: 1, 3, 5, 7, 9, 11, 13, 15, 17, 19, and 21, particularly SEQ ID NOs: 3, 5, 9, and 21, more particularly SEQ ID NO: 9.and 21. In other particular embodiments, the VH domain is a variant of a VH domain selected from SEQ ID NOs: 2, 4, 6, 8, 10, 12, 14, 16, 18, 20, and 22, particularly SEQ ID NOs: 4, 6, 10, and 22, more particularly SEQ ID NO: 10 and 22, and/or the VL domain is a variant of a VL domain selected from SEQ ID NOs: 1, 3, 5, 7, 9, 11, 13, 15, 17, 19, and 21, particularly SEQ ID NOs: 3, 5, 9, and 21, more particularly SEQ ID NO: 9 and 21, particularly a variant comprising one or more amino acid residue exchanges in the framework domains and/or in CDR residues not involved in antigen binding.

In particular embodiments, said isolated antibody or functional fragment thereof comprises an antigen-binding region comprising a VH/VL domain combination selected from SEQ ID NO: 1/SEQ ID NO: 2; SEQ ID NO: 3/SEQ ID NO: 4; SEQ ID NO: 5/SEQ ID NO: 6; SEQ ID NO: 7/SEQ ID NO: 8, SEQ ID NO: 9/SEQ ID NO: 10, SEQ ID NO: 11/SEQ ID NO: 12, SEQ ID NO: 13/SEQ ID NO: 14, SEQ ID NO: 15/SEQ ID NO: 16, SEQ ID NO: 17/SEQ ID NO: 18, and SEQ ID NO: 19/SEQ ID NO: 20, particularly SEQ ID NO: 3/SEQ ID NO: 4; SEQ ID NO: 5/SEQ ID NO: 6; and SEQ ID NO: 9/SEQ ID NO: 10, more particularly SEQ ID NO: 9/SEQ ID NO: 10. In particular other embodiments, said isolated antibody or functional fragment thereof comprises an antigen-binding region comprising a variant of a VHNL domain combination selected from SEQ ID NO: 1/SEQ ID NO: 2; SEQ ID NO: 3/SEQ ID NO: 4; SEQ ID NO: 5/SEQ ID NO: 6; SEQ ID NO: 7/SEQ ID NO: 8, SEQ ID NO: 9/SEQ ID NO: 10, SEQ ID NO: 11/SEQ ID NO: 12, SEQ ID NO: 13/SEQ ID NO: 14, SEQ ID NO: 15/SEQ ID NO: 16, SEQ ID NO: 17/SEQ ID NO: 18, and SEQ ID NO: 19/SEQ ID NO: 20, particularly SEQ ID NO: 3/SEQ ID NO: 4; SEQ ID NO: 5/SEQ ID NO: 6; and SEQ ID NO: 9/SEQ ID NO: 10, more particularly SEQ ID NO: 9/SEQ ID NO: 10, wherein in such variant at least the VL or the VH domain is a variant of the VL/VH domain listed.

In a particular embodiment, said isolated antibody or functional fragment thereof comprises an antigen-binding region comprising the VHNL domain combination SEQ ID NO: 21/SEQ ID NO: 22. In another embodiment, said isolated antibody or functional fragment thereof comprises a variant of the antigen-binding region comprising the VHNL domain combination SEQ ID NO: 21/SEQ ID NO: 22, wherein in such variant at least the VL or the VH domain is a variant of the VL/VH domain listed.

In particular embodiments, said isolated antibody or functional fragment thereof comprises an antigen-binding region that is a variant of the sequences disclosed herein. Accordingly, the invention includes isolated antibody or functional fragment thereof having one or more of the properties of the isolated antibody or functional fragment thereof comprising SEQ ID NOs: 1 to 20, particularly the properties defined in Sections [0042], [0047], and [0054] to [0061], comprising a heavy chain amino acid sequence with: at least 60 percent sequence identity in the CDR regions with the CDR regions comprised in SEQ ID NO: 2, 4, 6, 8; 10, 12, 14, 16, 18, or 20, particularly SEQ ID NOs: 4, 6, and 10, more particularly SEQ ID NO: 10, particularly at least 70 percent sequence identity, more particularly at least 80 percent sequence identity, and most particularly at least 90 percent sequence identity, and/or at least 80 percent sequence homology, more particularly at least 90 percent sequence homology, most particularly at least 95 percent sequence homology in the CDR regions with the CDR regions comprised in SEQ ID NO: 2, 4, 6, 8; 10, 12, 14, 16, 18, or 20, particularly SEQ ID NOs: 4, 6, and 10, more particularly SEQ ID NO: 10, and/or comprising a light chain amino acid sequence with: at least 60 percent sequence identity in the CDR regions with the CDR regions comprised in SEQ ID NO: 1, 3, 5, 7; 9, 11, 13, 15, 17, or 19, particularly SEQ ID NOs: 3, 5, and 9, more particularly SEQ ID NO: 9, particularly at least 70 percent sequence identity, more particularly at least 80 percent sequence identity, and most particularly at least 90 percent sequence identity, and/or at least 80 percent sequence homology, more particularly at least 90 percent sequence homology, most particularly at least 95 percent sequence homology in the CDR regions with the CDR regions comprised in SEQ ID NO: 1, 3, 5, 7; 9, 11, 13, 15, 17, or 19, particularly SEQ ID NOs: 3, 5, and 9, more particularly SEQ ID NO: 9. Methods for the determination of sequence homologies, for example by using a homology search matrix such as BLOSUM (Henikoff, S. & Henikoff, J. G. (1992). Amino acid substitution matrices from protein blocks. Proc. Natl. Acad. Sci. USA 89, 10915-10919), and methods for the grouping of sequences according to homologies are well known to one of ordinary skill in the art.

In particular embodiments, such a variant comprises a VL sequence comprising the set of CDR1, CDR2 and CDR3 sequences according to the VL sequence of SEQ ID NO: 19, and/or a VH sequence comprising the set of CDR1, CDR2 and CDR3 sequences according to the VH sequence of SEQ ID NO: 20, wherein in each case one of the indicated amino acid residues shown at every degenerate position “X” in SEQ ID NO: 19 and/or 20 is selected. For example, in the case of each of the positions shown as “X(S/N)” in the CDR1 of SEQ ID NO: 19, any such variant comprises either amino acid residue “S” or amino acid residue “N” at the corresponding positions.

In particular other embodiments, such a variant comprises a VL sequence according to the sequence of SEQ ID NO: 19, and/or a VH sequence according to the sequence of SEQ ID NO: 20, wherein in each case one of the indicated amino acid residues shown at every degenerate position “X” in SEQ ID NO: 19 and/or 20 is selected. For example, in the case of the position shown as “X(P/A)” in framework 1 of SEQ ID NO: 19, any such variant comprises either amino acid residue “P” or amino acid residue “A” at that position.

In particular embodiments, said isolated antibody or functional fragment thereof comprises an antigen-binding region which is obtained by humanizing an antigen-binding region of Sections [0078] to [0080], and [0083] to [0085]

In a seventh aspect, the present invention relates to an isolated antibody or functional fragment thereof binding to essentially the same epitope as the isolated antibody or functional fragment thereof of Sections [0078] to [0080], [0083] to [0085] and [0089].

In particular embodiments, said isolated antibody or functional fragment thereof is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

In an eighth aspect, the present invention relates to a pharmaceutical composition comprising a binding molecule of the present invention, in particular an isolated antibody or functional fragment thereof, and optionally a pharmaceutically acceptable carrier and/or excipient.

In a ninth aspect, the present invention relates to a nucleic acid sequence or a collection of nucleic acid sequences encoding a binding molecule of the present invention, in particular an isolated antibody or functional fragment thereof.

In a tenth aspect, the present invention relates to a vector or a collection of vectors comprising the nucleic acid sequence or a collection of nucleic acid sequences of the present invention.

In an eleventh aspect, the present invention relates to a host cell, particularly an expression host cell, comprising the nucleic acid sequence or the collection of nucleic acid sequences of the present invention, or the vector or collection of vectors of the present invention.

In a twelfth aspect, the present invention relates to a method for producing a binding molecule of the present invention, in particular an isolated antibody or functional fragment thereof, comprising the step of expressing the nucleic acid sequence or the collection of nucleic acid sequences of the present invention, or the vector or collection of vectors of the present invention, or the host cell, particularly an expression host cell, of the present invention.

In a thirteenth aspect, the present invention relates to a method for generating an isolated antibody or functional antibody fragment in accordance with the present invention comprising the steps of:
a) Immunization of rabbits with a CD3ε-expressing plasmid to present the native full-length CD3ε on the surface of host cells;
b) Clonal isolation of affinity matured memory B-cells that interact with the CD3ε/γ single-chain, preferably using fluorescence activated cell-sorting;
c) Cultivation of single sorted B cells, preferably in a co-cultivation system that does not require immortalization of sorted clones;. p0 d) Screening of B cell culture supernatants to identify antibodies binding to the native CD3ε embedded in the TCR complex on the surface of T cells, particularly by a cell-based ELISA.

In a fourteenth aspect the present invention relates to a particular epitope of human CD3 epsilon comprising exclusively amino acid residues of CD3 epsilon that are not located in the interface between CD3 epsilon and CD3 gamma and that still can be bound by an antibody in the context of the native TCR expressed on T cells, binding of which by a cross-linked antibody of the invention is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; and/or (iic) is resulting in a dose-dependent homogeneous activation state of T-cells.

In a fifteenth aspect, the present invention relates to a method for identifying a binding molecule comprising a binding region that is specific for a novel epitope of human CD3ε, comprising the step of (a) selecting from one or more molecules binding to human CD3 at least one binding molecule, which comprises a binding region that is specific for an epitope of human CD3ε, wherein said epitope comprises amino acid residue N4 as residue that is critical for binding.

In particular embodiments, step (a) is performed by performing an epitope mapping using overlapping peptides spanning the N-terminal part of CD3ε, to identify the critical linear binding region of the respective CD3ε-binder. In step (b) derivatives of this linear binding region are generated in which at each position individually, the wild-type amino acid is exchanged by either (i) alanine, or (ii) any of the natural amino acids (except cysteine) separately. The resulting peptide library is screened by use of the ELISA described in Examples 7 and 8 to assess the relevance of each position for binding. In particular, a set of peptides is used that is selected from the list of:

### EXAMPLES

The following examples illustrate the invention without limiting its scope.

The approach used for the invention described herein is a step-wise procedure to increase the probability of success to identify T cell stimulatory antibodies. This approach encompasses the following procedure:
a) Using rabbits as a host for immunization, as rabbit antibodies generally show greater clonal diversity as compared to rodents. Therefore, the use of rabbits increases the probability to identify binders against a particular epitope and enhances the probability of identifying novel epitopes.
b) Immunizing rabbits with a CD3ε-expressing plasmid to present the native full-length CD3ε on the surface of host cells. This approach leads to a strong immune response against full-length CD3ε and avoids the generation of antibodies concomitantly binding to CD3ε and CD3γ;
c) Clonal isolation of affinity matured memory B-cells that interact with the CD3ε/γ single-chain using fluorescence activated cell-sorting. This procedure avoids the selection of antibodies binding to the interface between CD3ε and CD3γ, thereby increasing the specificity of the selection.
d) Cultivation of single sorted B cells in a co-cultivation system that does not require immortalization of sorted clones, thereby overcoming the poor efficiency of the hybridoma procedure.
e) Screening of B cell culture supernatants in a cell-based ELISA to identify antibodies binding to the native CD3ε embedded in the TCR complex on the surface of T cells.

### Example 1

### Identification and Selection of Monoclonal Antibodies Binding to a T Cell-Stimulatory Epitope on CD3

Rabbit memory B cells binding to CD3ε were isolated from one immunized rabbit using fluorescence activated cell sorting. In order to exclude antibodies binding to the interface of CD3ε and CD3γ, a Phycoerythrin (PE)-labeled single-chain protein construct was used consisting of the extracellular domains of CD3ε and CD3γ joined by a flexible peptide linker (scCD3γε). In total, 4,270 memory B cells binding to PE-scCD3γε were individually sorted into 96-well culture plates and cultured at conditions published elsewhere (Lightwood et al, JIM 2006; 316: 133-143). All culture supernatants were first screened by ELISA for binding to scCD3γε, which yielded 441 hits. In a second screening, positive supernatants from the first screening were tested for their ability to bind the native CD368 embedded in the TCR complex on the surface of Jurkat cells (see Methods below). A total of 22 hits showed binding to CD3ε expressing Jurkat cells but not to cd3−/− Jurkat cells. The affinity to the purified extracellular domain of heterodimeric CD3εγ from human and cynomolgus monkey origin was measured using SPR for the 22 hits. Affinities to human CD3εγ as expressed by K_{D} ranged from 0.16 to 9.28 nM (data not shown). One of the screening hits did not show binding by SPR and was therefore not considered for further analysis.

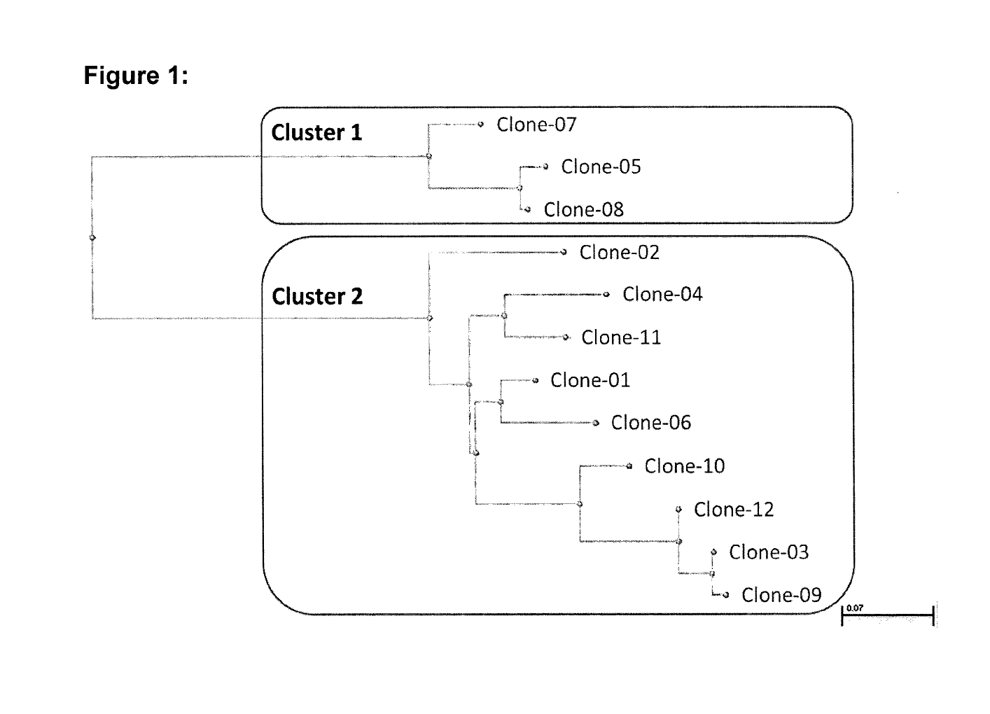

The DNA sequence encoding the variable domains of the remaining 21 clones were retrieved by RT-PCR and DNA sequencing and resulted in 18 independent clones. These rabbit IgGs were recombinantly produced in a mammalian expression system and were characterized in terms of affinity to scCD3γε from human and cynomolgus origin and their ability to bind to Jurkat cells. Phylogenetic sequence analysis of these 18 sequences revealed two main clusters, which clearly differed from each other, while there was significant homology within the two clusters (FIG. 1). As all representatives from one cluster presumably derive from the same antigen-binding parent B cell they likely bind to the same epitope. Thus, in order to cover the maximal diversity, the most diverse clones were selected from each cluster resulting in 12 clones that were further tested for their ability to bind and activate T cells. T cell binding was assessed in a cell-based ELISA and T cell stimulation was quantified by measuring expression of CD69 by FACS. Representative antibodies were further characterized as shown in Examples 2 to 4.

### Example 2

### Binding of Purified Monoclonal Rabbit Anti-CD3ε Antibodies to Jurkat T Cells and to Cynomolgus Monkey HSC-F T Cells

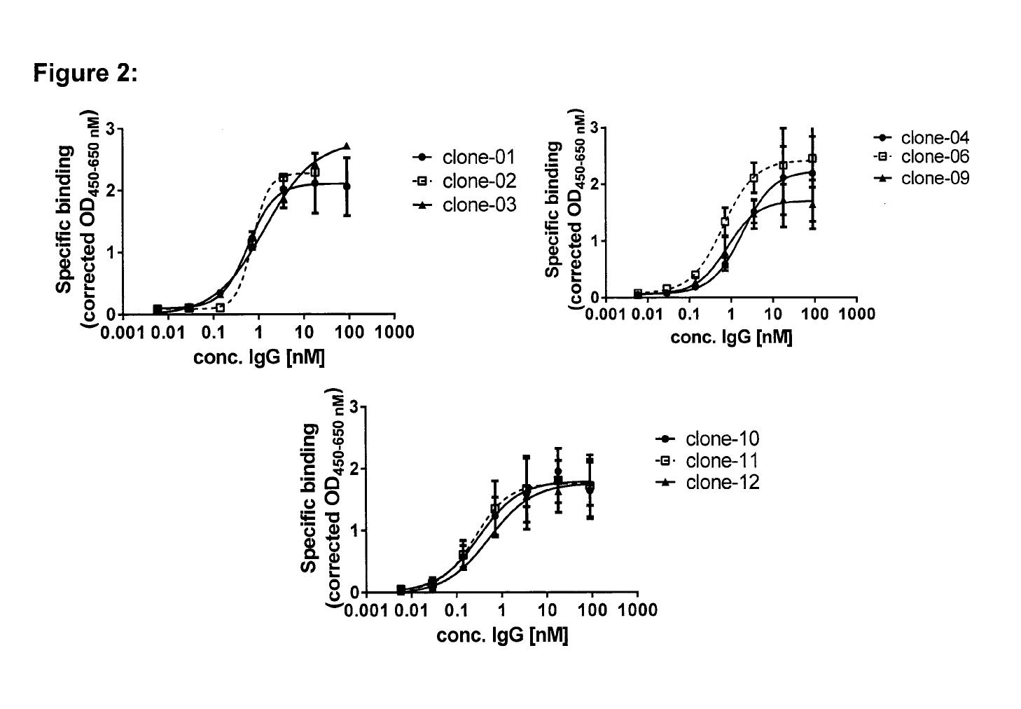

Jurkat human T cells and cynomolgus monkey HSC-F T cells were incubated with increasing concentrations of the purified monoclonal rabbit antibodies, as described in the methods section. With all antibodies tested, specific binding to human CD3ε increased with increasing antibody concentrations (FIG. 2). The EC₅₀ values, indicating half-maximal binding to Jurkat human T cells, were very similar for all antibodies, ranging from 0.28 to 1.87 nM (see Table 1, which shows the pharmacodynamic characteristics of purified monoclonal rabbit antibodies. For the qualitative detection of CD69 expression the mean fluorescence intensity (MFI), reflecting the signal intensity at the geometric mean, was measured for both, the negative control as well as for the test antibodies. The normalized MFI was calculated by dividing the MFI of the test antibody through the MFI of the negative control antibody.). EC₅₀ values for binding to cynomolgus monkey HSC-F T cells are shown for 3 antibodies (clone-06, clone-02, clone-03) (see Table 2C).

TABLE 1

| | | | | | | | | | Fold increase in CD69 expression: | | |
|---|---|---|---|---|---|---|---|---|---|---|---|
| | | | | | | | Specific binding | | [MFI normalized to neg. ctrl.] | | |
| | SPR data human CD3ge | | | SPR data cyno CD3ge | | | to Jurkat cells | | 20 μg/ml | 5 μg/ml | 1.25 μg/ml |
| | ka [M⁻¹ | | KD | ka [M⁻¹ | | KD | EC50 | relative EC₅₀ | anti-CD3 | anti-CD3 | anti-CD3 |
| Clone ID | s⁻¹] | kd [s⁻¹] | [M] | s⁻¹] | kd [s⁻¹] | [M] | (nM) | (EC_{50,clone 5}/EC_{50,test)} | IgG | IgG | IgG |
| clone-01 | 5.36E+05 | 1.59E−03 | 2.97E−09 | 3.86E+05 | 3.92E−03 | 1.02E−08 | 0.58 | 0.88 | ND | ND | ND |
| clone-02 | 8.69E+05 | 2.64E−04 | 3.04E−10 | 6.68E+05 | 2.58E−03 | 3.86E−09 | 0.71 | 0.59 | 7.4 | 4.6 | 3.3 |
| clone-03 | 5.51E+05 | 4.98E−04 | 9.05E−10 | 3.50E+05 | 4.03E−03 | 1.15E−08 | 1.45 | 0.37 | 6.6 | 4.6 | 2.6 |
| clone-04 | 8.73E+05 | 9.88E−05 | 1.13E−10 | 6.46E+05 | 2.66E−03 | 4.12E−09 | 1.87 | 0.29 | 7.8 | 3.5 | 2.6 |
| clone-06 | 6.18E+05 | 1.38E−03 | 2.23E−09 | 4.44E+05 | 3.97E−03 | 8.95E−09 | 0.67 | 0.76 | 5.3 | 5.1 | 2.7 |
| clone-09 | 6.01E+05 | 6.88E−04 | 1.14E−09 | 2.32E+05 | 2.69E−03 | 1.16E−08 | 0.82 | 0.90 | ND | ND | ND |
| clone-10 | 7.57E+05 | 1.26E−03 | 1.66E−09 | 3.21E+05 | 3.49E−03 | 1.09E−08 | 0.35 | 2.10 | 6.2 | 4.2 | 2.6 |
| clone-11 | 4.25E+05 | 1.33E−03 | 3.13E−09 | 3.63E+05 | 3.65E−03 | 1.00E−08 | 0.28 | 2.39 | ND | ND | ND |
| clone-12 | 7.21E+05 | 7.98E−04 | 1.11E−09 | 1.42E+05 | 3.14E−03 | 2.22E−08 | 0.59 | 1.14 | ND | ND | ND |

| | | | | | | |
|---|---|---|---|---|---|---|
| OKT3 | ND | ND | ND | 3.1 | 2.5 | 1.8 |
| TR66 | ND | ND | ND | 3.0 | 2.2 | 1.6 |

TABLE 2A

| Clone ID | KD (human)/KD (cyno) |
|---|---|
| clone-01 | 3 |
| clone-02 | 13 |
| clone-03 | 13 |
| clone-04 | 36 |
| clone-06 | 4 |
| clone-09 | 10 |
| clone-10 | 7 |
| clone-11 | 3 |
| clone-12 | 20 |

TABLE 2B

| | Affinity to human CD3ε | Affinity to cyno CD3ε |
|---|---|---|
| Clone ID | [KD] | [KD] |
| clone-06 | 2.23 × 10⁻⁹M | 8.95 × 10⁻⁹M |
| clone-02 | 3.04 × 10⁻¹⁰M | 3.86 × 10⁻⁹M |
| clone-03 | 9.05 × 10⁻¹⁰M | 1.15 × 10⁻⁸M |

TABLE 2C
Rabbit IgG binding to cell surface

| | Binding to human Jurkat | Binding to cyno HSC-F T |
|---|---|---|
| Clone ID | T cells [EC₅₀] | cells [EC₅₀] |
| clone-06 | 0.67 nM | 1.6 nM |
| clone-02 | 0.71 nM | 3.82 nM |
| clone-03 | 1.45 nM | 23.9 nM |

### Example 3

### Potential of Purified Monoclonal Rabbit Anti-CD3E Antibodies to Stimulate CD69 Expression on T Cells

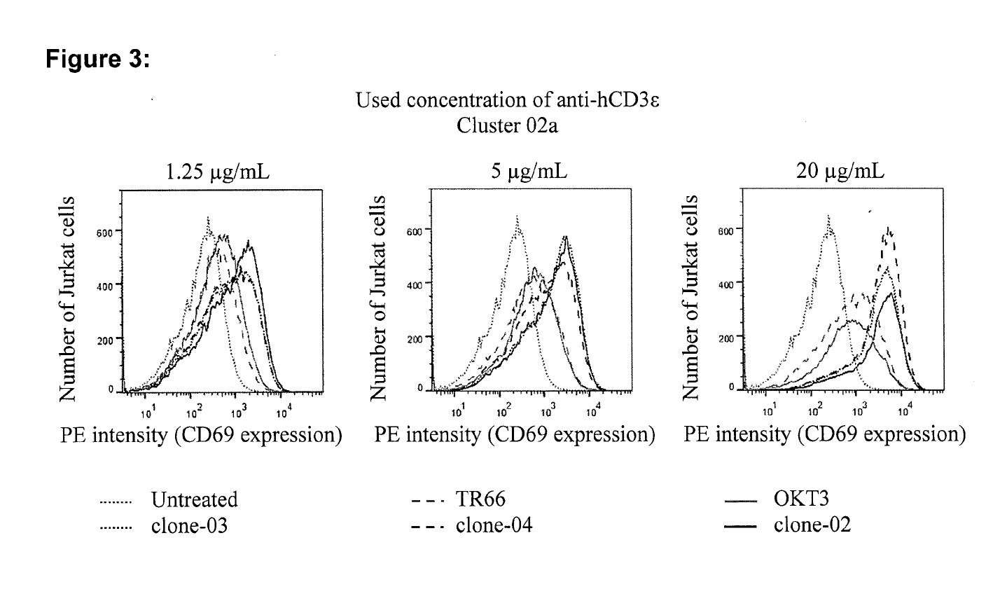

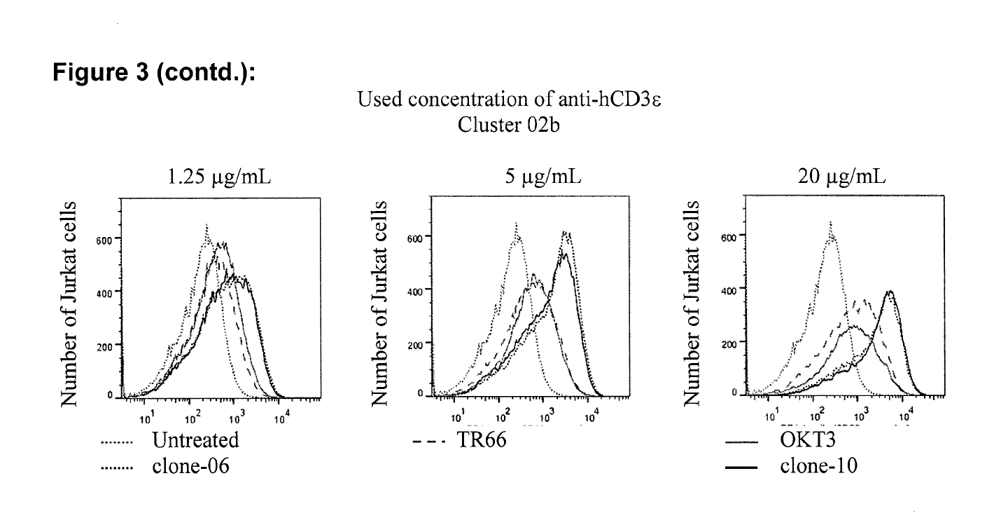

The potential of purified monoclonal rabbit anti-CD3 antibodies to induce T-cell activation as assessed by measurement of CD69 expression (see methods) was compared to the published antibodies OKT-3 and TR66. In the first approach, three different concentrations of cross-linked antibodies were used to stimulate Jurkat cells and CD69 expression was assessed by flow-cytometry 24 h later. A significant increase in CD69 expression was observed with all tested antibodies at 1.25 μg/ml (FIG. 3 and Table 1). Interestingly, all tested rabbit antibodies showed stronger stimulation of CD69 expression than the published OKT-3 and TR66. This is unexpected as the rabbit antibodies bind to human scCD3γε with much higher affinity than OKT-3 or TR66, which, according to prior art, should negatively affect their ability to serially trigger and thereby enhance TCR signaling. With increasing concentrations of rabbit antibodies the CD69 expression level further increased, while there was only a moderate increase in CD69 expression with increasing concentrations of OKT-3 or TR66. Further with the rabbit antibodies, the peak in the histogram became narrower indicating a more homogenous population of T cells, all expressing CD69 at similarly high levels. In contrast there were broad distributions of CD69 expression levels in the T cell populations stimulated with OKT-3 or TR66 at each concentration tested. An antibody that leads to distinct and homogenous T cell activation levels depending on the dose allows for better dose adjustment to optimize efficacy and to control side effects.

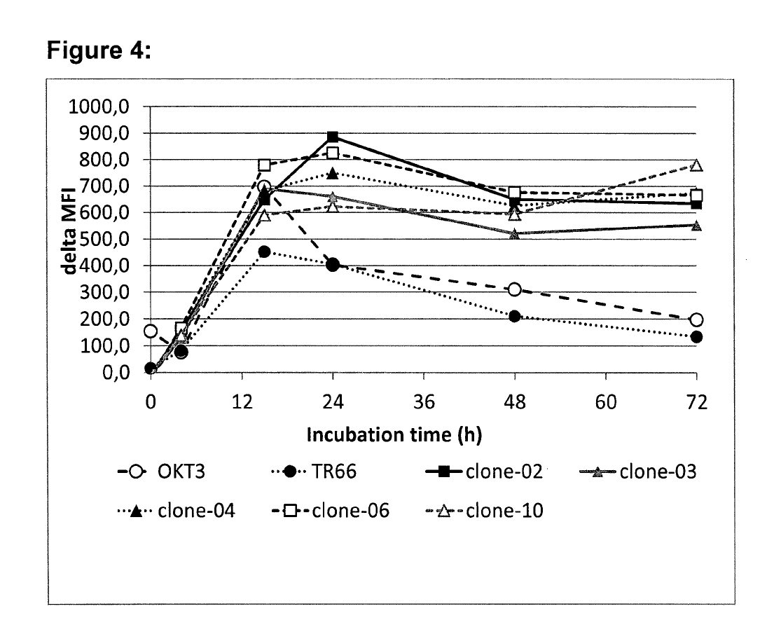

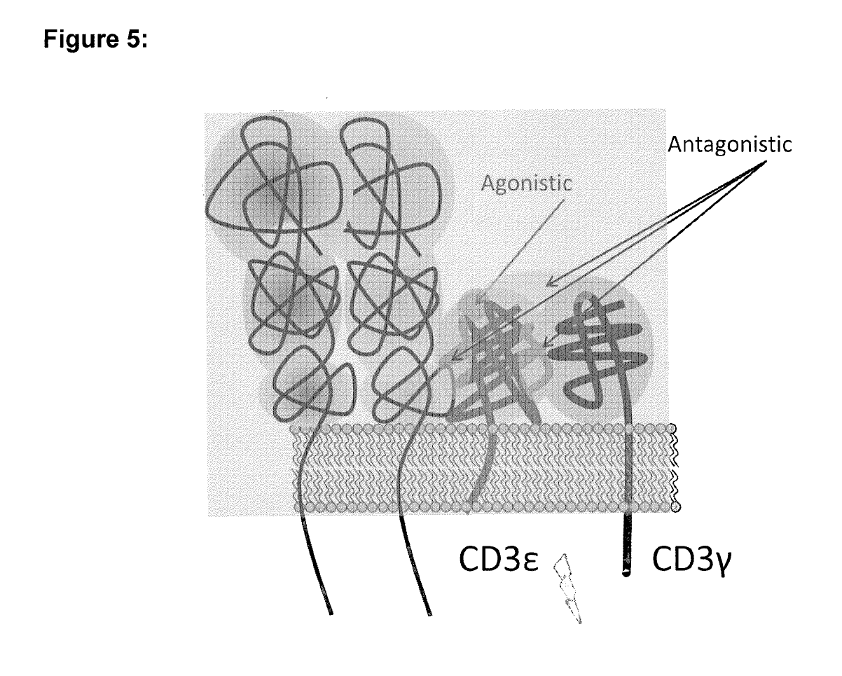

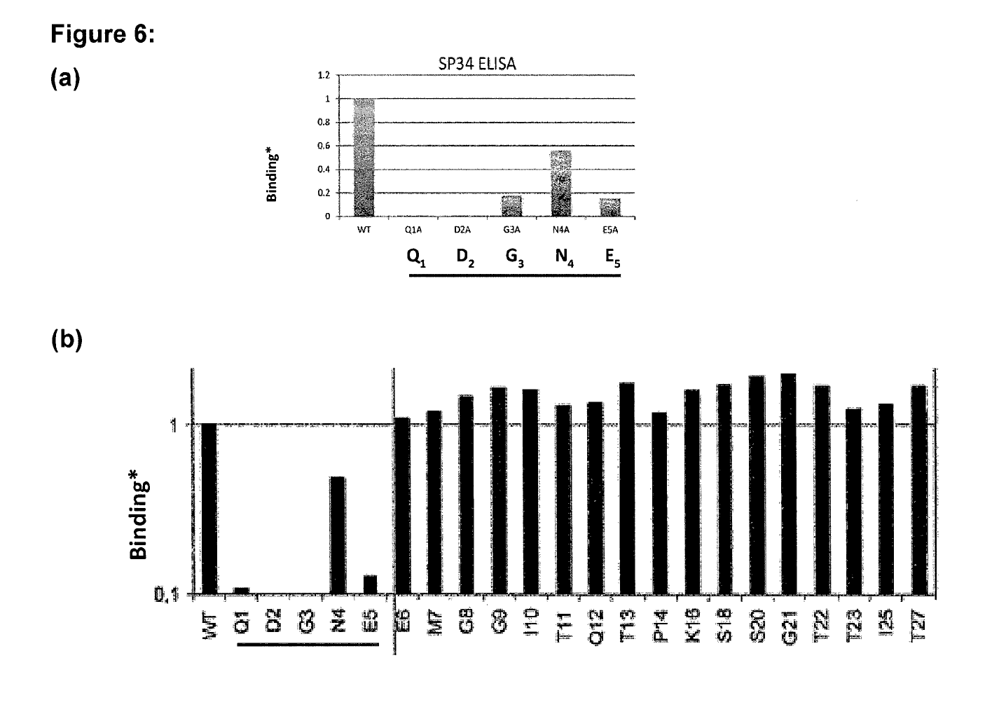

In the second approach, T-cell activation after different time points of stimulation by anti-CD3 antibodies was analyzed. Jurkat cells were stimulated by cross-linked antibodies and CD69 expression was assessed as described above after 0, 4, 15, 24, 48 and 72 h (FIG. 4).

### Example 4

### Binding of Anti-CD3×Anti-IL5R Antibodies to Jurkat T Cells and CHO-IL5R Cells

In order to show the benefit of the agonistic anti-CD3 antibodies, a set of bispecific anti-CD3×IL5R single-chain diabodies (scDbs) were constructed by standard methods (methods/data not shown; Construct 1=comprises the humanized variable domain of clone-06; Construct 2=comprises the humanized variable domain of clone-02; Construct 3=comprises the humanized variable domain of clone-03).

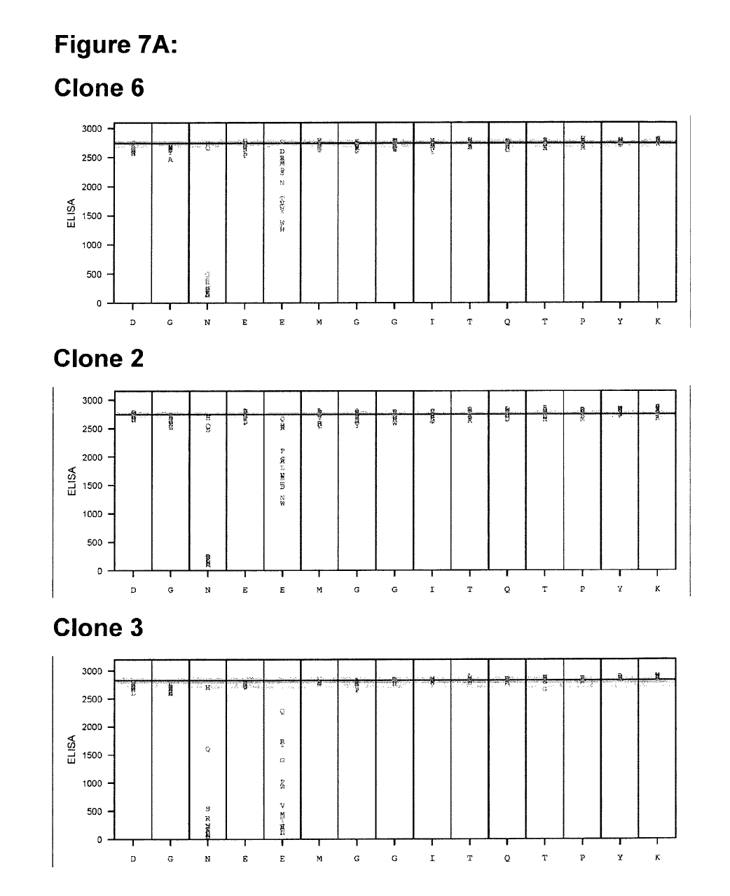

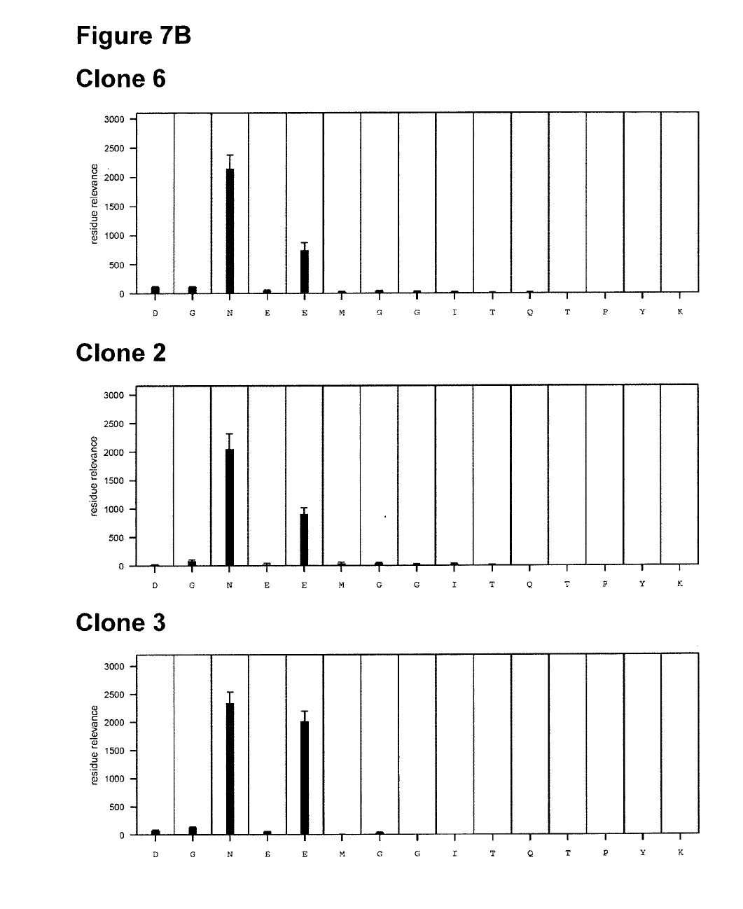

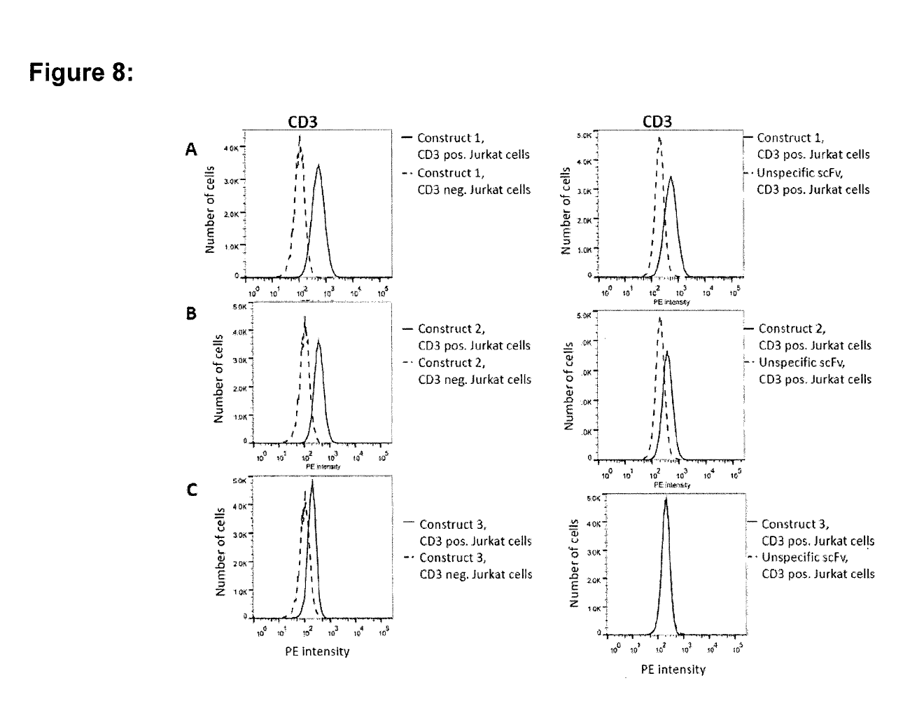

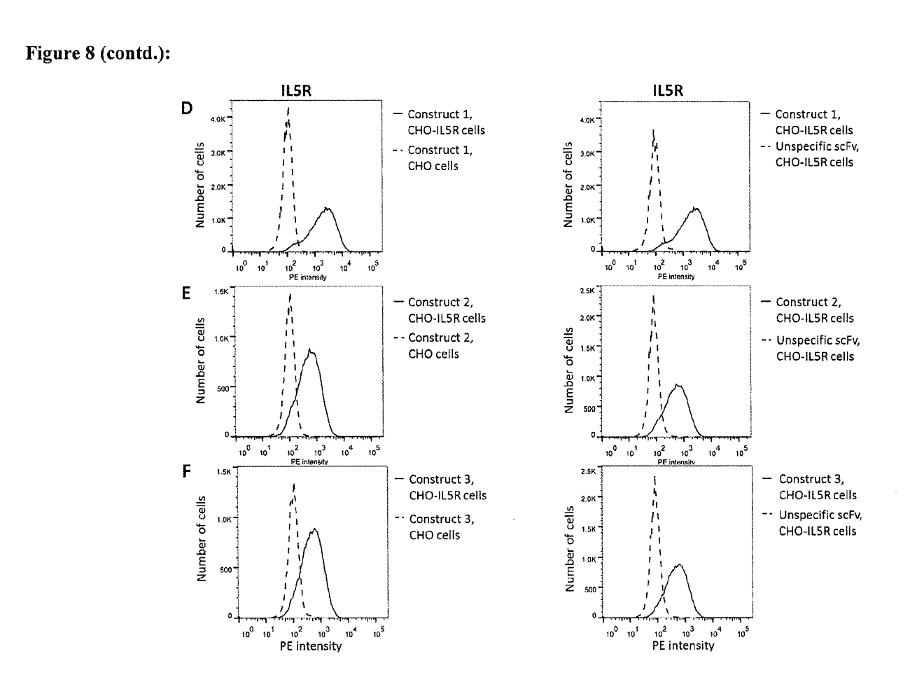

Jurkat T cells and IL5R-expressing CHO cells (CHO-IL5R) are incubated with 1 μg/ml and 10 μg/ml of the scDbs, as described in the methods section. With all scDbs tested, specific binding to CD3ε and IL5R expressing cell lines but no unspecific binding to control cell lines is detected. The three different scDbs (Constructs 1 to 3) containing the identical anti-IL5R moiety while the anti-CD3 moieties being different, were tested for specific binding to cells expressing either IL5R or CD3ε. The anti-CD3 parts bind to overlapping epitopes with variable affinities though (Table 1 and 3 and FIG. 7). As expected the binding to CHO-IL5R cells was similar for all scDbs tested (FIG. 8). In contrast, binding to Jurkat T-cells decreased with decreasing affinity of the CD3ε binding domain. No binding to Jurkat T-cells was detected for the low affinity binder Construct 3 at the highest concentration tested (FIG. 8).

### Example 5

### Potential of Bispecific anti-CD3×IL5R scDbs to Stimulate IL-2 Secretion from T Cells

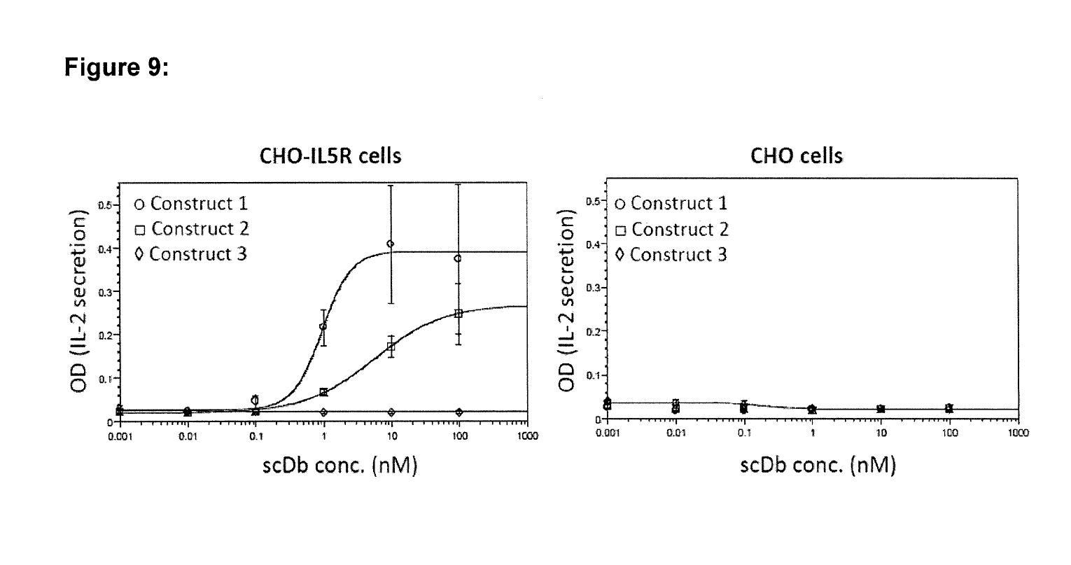

The potential of scDbs bound to a target cell to induce T-cell activation can be assessed by measurement of IL-2 secretion (see methods) by cytotoxic T-cells purified from human blood. The different scDbs are incubated with CD8+ cytotoxic T-cells in presence of target expressing CHO-IL5R cells at an effector:target cell ratio of 10:1 and IL-2 secretion is analysed after 16 hours of incubation. A dose-dependent stimulation of IL-2 secretion is observed in presence of CHO-IL5R cells while essentially no IL-2 secretion is observed in presence of wild-type CHO cells (see representative data in Table 3 and in FIG. 9). Therefore, T-cell activation is specifically induced in presence of target expressing cells. Moreover, the potential to induce IL-2 secretion correlates with binding affinity to recombinantly produced CD3εγ and to the capacity to bind to T-cells. In line with affinity analysis, Construct 1, which is the binder with the highest affinity, is a more potent inducer of IL-2 secretion than Construct 2, while no IL-2 secretion is observed with the low affinity scDb Construct 3 (FIG. 9).

TABLE 3
Humanized anti-CD3ε domains in the IL5R×CD3 scDb format

| | | | Potency to |
|---|---|---|---|
| | Affinity human | | stimulate IL-2 |
| | CD3ε | Potency to lyse | secretion by T cells |
| Clone ID | [KD] | target cells [EC₅₀] | [EC₅₀] |
| clone-06 | 1.15 × 10⁻⁸M | 0.1 nM | 0.96 nM |
| clone-02 | 2.96 × 10⁻⁸M | 0.96M | 5.67 nM |
| clone-03 | 1.23 × 10⁻⁷M | no lysis | no signal |

### Example 6

### Specific scDb Mediated Target Cell Lysis by Cytotoxic T-Cells

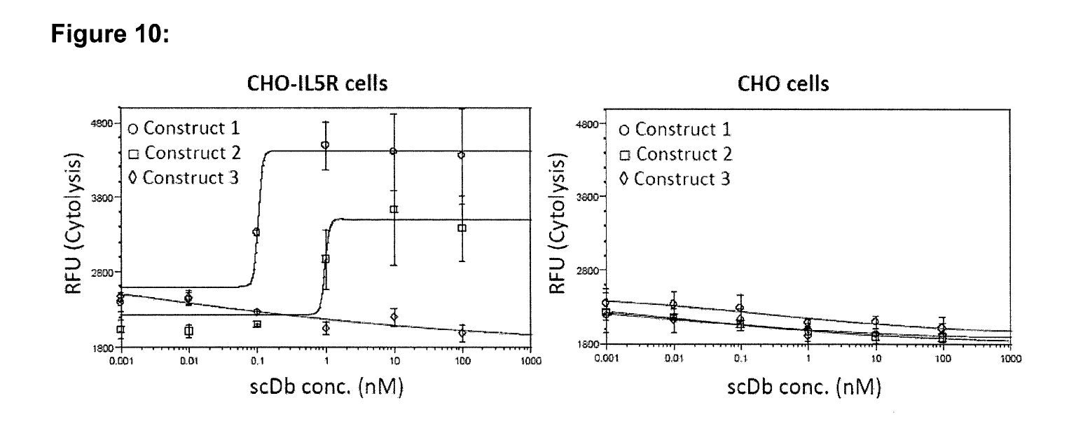

Specific lysis of target cells by cytotoxic T-cells mediated by anti-CD3×IL5R scDbs is analyzed with the CellTox™ green cytotoxicity assay (see methods) after 88 hours of incubation. Similarly to results discussed above for T-cell activation, a dose-dependent target cell lysis is observed for Construct 1 and Construct 2 in presence of CHO-IL5R cells while no lysis is observed in presence of wild-type CHO cells (see representative data for constructs 1 to 3 in Table 3 and in FIG. 10). In line with results mentioned above, scDbs binding with high affinity to CD3ε shows more potent lysis compared to the lower affinity scDbs. No target cell lysis is observed for the low affinity scDb Construct 3. We further tested the potency to lyse target cells of a scDb containing the humanized variant of an additional CD3ε binding clone (clone-05) originating from a different cluster (cluster 1) of antibodies that are also cross-reactive to cynomolgus monkey CD3ε. Clone-05 binds with even higher affinity (KD=8.45×10⁻¹⁰ M and 4.29×10⁻⁹ M for the rabbit IgG and humanized derivative thereof, respectively) as compared to clone-06, but importantly, binds to a different epitope than the binders from cluster 2 (clone-06, clone-02 and clone-03). Interestingly, we found that the respective anti-IL5R×CD3 scDb showed weaker potency to induce T-cell dependent lysis of CHO-IL5R target cells (EC50=9.9×10⁻⁹ M) than the scDb containing humanized clone-06, suggesting that the epitope of clone-06 is particularly well suited for the redirection of cytotoxic T cells to lyse target cells. The superior potency of the cross-linked parental IgGs from cluster 2 versus OKT-3 and TR66 (example 3) confirm that even with higher affinities than those tested in the scDb format no affinity optimum was found after which the potency would decrease.

### Example 7

### Epitope Mapping and Fine-Mapping

Epitope mapping and fine-mapping were performed essentially as described (Timmerman et al., Functional reconstruction and synthetic mimicry of a conformational epitope using CLIPS™ technology.J.Mol.Recognit.20 (2007) 283-99; Slootstra et al., Structural aspects of antibody antigen interaction revealed through small random peptide libraries, Molecular Diversity 1: 87 (1996) 96). In brief, CLIPS technology structurally fixes peptides into defined three-dimensional structures. This results in functional mimics of even the most complex binding sites. CLIPS technology is now routinely used to shape peptide libraries into single, double or triple looped structures as well as sheet and helix-like folds.

CLIPS library screening starts with the conversion of the target protein into a library of up to 10,000 overlapping peptide constructs, using a combinatorial matrix design. On a solid carrier, a matrix of linear peptides is synthesized, which are subsequently shaped into spatially defined CLIPS constructs. Constructs representing both parts of the discontinuous epitope in the correct conformation bind the antibody with high affinity, which is detected and quantified. Constructs presenting the incomplete epitope bind the antibody with lower affinity, whereas constructs not containing the epitope do not bind at all. Affinity information is used in iterative screens to define the sequence and conformation of epitopes in detail.

The following clones were analyzed: clone-02, clone-03, clone-04, clone-06, and clone-10. The following target sequences of CD3 (N-terminal sequences) were used

| HumanCD3: | | |
|---|---|---|
| 2 | DGNEEMGGIT QTPYKVSISG TTVILTCPQY PGSEILWQHN DKNIGGDEDD | 51 |
| 52 | KNIGSDEDHL SLKEFSELEQ SGYYVCYPRG SKPEDANFYL YLRARVCENC | 101 |
| 102 | MEMD | 105 |
| Cynomolgus CD3: | | |
| 2 | DGNEEMGSIT QTPYQVSISG TTVILTCSQH LGSEAQWQHN GKNKEDSGDR | 51 |
| 52 | LFLPEFSEME QSGYYVCYPR GSNPEDASHH LYLKARVCEN CMEMD | 96 |

### Sequence Alignments

CLUSTAL 2.1 multiple sequence alignment:

| | |
|---|---|
| Human | DGNEEMGGITQTPYKVSISGTTVILTCPQYPGSEILWQHNDKNIGGDEDDKNIG |
| | \|\|\|\|\|\|\|.\|\|\|\|\|\|:\|\|\|\|\|\|\|\|\|\|\|\|.\|: \|\|\| \|\|\|\|.\|\| \|\|. \| |
| Cynomolgus | DGNEEMGSITQTPYQVSISGTTVILTCSQHLGSEAQWQHNGKNK---EDS---G |
| Human | SDEDHLSLKEFSELEQSGYYVCYPRGSKPEDANFYLYLRARVCENCMEMD |
| | \|:\| \| \|\|\|\|:\|\|\|\|\|\|\|\|\|\|\|\|\|:\|\|\|\| :\|\|\|:\|\|\|\|\|\|\|\|\|\|\| |
| Cynomolgus | ---DRLFLPEFSEMEQSGYYVCYPRGSNPEDASHHLYLKARVCENCMEMD |

### Synthesis of Peptides

To reconstruct discontinuous epitopes of the target molecule a library of structured peptides was synthesized. This was done using the so-called “Chemically Linked Peptides on Scaffolds” (CLIPS) technology. CLIPS technology allows structuring peptides into single loops, double loops, triple loops, sheet like folds, helix like folds and combinations thereof. CLIPS templates are coupled to cysteine residues. The side chains of multiple cysteines in the peptides are coupled to one or two CLIPS templates. For example, a 0.5 mM solution of the T2 CLIPS template 1,3 bis (bromomethyl) benzene is dissolved in ammonium bicarbonate (20 mM, pH 7.9)/acetonitrile (1:1(v/v). This solution is added onto the peptide arrays. The CLIPS template will bind to side chains of two cysteines as present in the solid phase bound peptides of the peptide arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt buffer containing 1 percent SDS/0.1 percent beta mercaptoethanol in PBS (pH 7.2) at 70° C. for 30 min, followed by sonication in H₂O for another 45 min. The T3 CLIPS carrying peptides were made in a similar way but now with three cysteines.

### ELISA Screening

The binding of antibody to each of arrays were incubated with primary antibody solution (overnight at 4° C.). After washing, the peptide arrays were incubated with a 1/1000 dilution of an antibody peroxidase conjugate (SBA, cat.nr.2010 05) for one hour at 25° C. After washing, the peroxidase substrate 2,2′ azino di 3 ethylbenzthiazoline sulfonate (ABTS) and 2 μl/ml of 3% H₂O₂ were added. After one hour, the color development was measured. The color development was quantified with a charge coupled device (CCD) camera and an image processing system.

### Design Of Peptides

Chemically synthesized CLIPS peptides were synthesized as described above according to the following designs.

### Set 1

Mimic Type Linear peptides: Double sets of linear peptides for both human and cynomolgus sequences. Length is 15 residues with an overlap of 14. Two of the sets feature a double alanine mutation (shown in grey).
Sequences (first 10 of human sequences shown)

```
  DGNEEMGGITQTPYK
    GNEEMGGITQTPYKV
      NEEMGGITQTPYKVS
        EEMGGITQTPYKVSI
          EMGGITQTPYKVSIS
            MGGITQTPYKVSISG
              GGITQTPYKVSISGT
                GITQTPYKVSISGTT
                  ITQTPYKVSISGTTV
                    TQTPYKVSISGTTVI

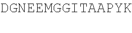

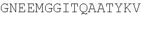

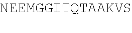

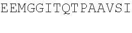

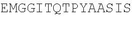

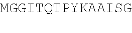

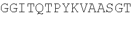

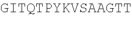

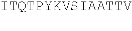

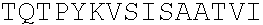










```

### Set 2

Mimic Type Linear peptides with added charges
Description Control sets with added charges that are required for some antibodies that strongly interact with the peptide array surface
Sequences (first 10 of human sequence shown)

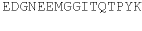

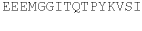

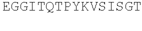

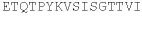

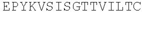

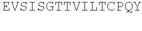

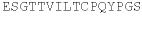

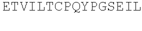

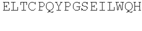

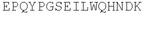

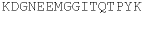

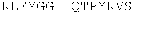

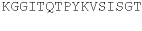

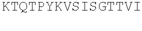

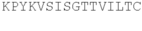

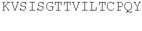

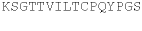

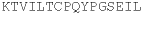

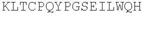

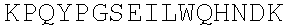

### Set 3

Mimic Type Conformational peptides
Description Peptide sequence are similar to Set 1, but are constrained into a CLIPS conformational loop.
Sequences (first 10 of unmodified human sequence shown)

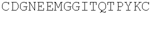

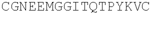

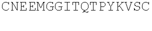

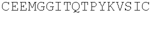

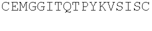

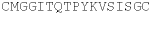

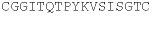

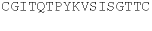

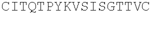

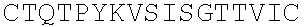

### Set 4

Mimic Type CLIPS conformational peptides
Description Overlapping set of 20mer CLIPS conformational peptides
Sequences (first 10 of human sequence shown)

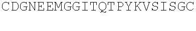

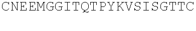

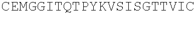

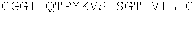

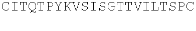

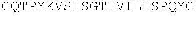

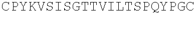

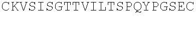

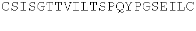

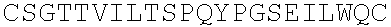

### Set 5

Mimic Type CLIPS discontinuous matrix peptides
Description Combinatorial set of 13mer peptides, constrained pairwise into a double looped CLIPS structure. Human and Cynomolgus peptides are ordered according to pairwise alignment to minimize technical variation.
Sequences (first 10 shown)

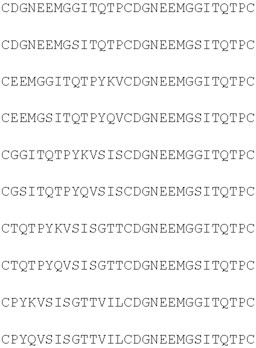

### Identification of Putative Epitopes

In general, all five antibodies showed very similar binding characteristics. All binding took place on the N terminus of human CDD3ε (data not shown). Considering the binding strength and observations from constrained and non-constrained peptides, it is most likely that all antibodies bind predominantly to linear epitopes as:
Binding was observed only to N-terminal sequences
Loss of D2 or G3 does not strongly reduce binding
Loss of 2DGN4 completely abolishes binding.

### Conclusion

The analysis identified binding regions for all five antibodies tested. All antibodies were found to bind to a seemingly linear epitope on the N terminus. All antibodies were found to bind to a similar epitope that relied strongly on 2DGN4 for binding.

### Example 8

### Epitope Fine-Mapping

### Methods

15mer linear arrays derived from human and cynomolgus CD3ε, residues 2-16 and 5-20, in which each position is substituted by 18 amino acids (all natural amino acids except cysteine) were probed with the antibodies and specificities affecting the binding were found.

### Results

All antibodies bind the N terminus with an absolute requirement for N4 and an involvement of E6, and share significant similarities. All antibodies bind both human and cynomolgus versions of CD3ε, despite the small differences in sequence adjacent to the core epitope.

### Target Protein

The initial mapping identified a linear stretch on the N terminus of CD3ε as the core epitope for all antibodies tested. Residues 2-20 of the sequences below were used to design full substitution libraries of linear 15mer peptides.

### Methods

### Synthesis of Peptides

Linear peptides were synthesized by standard Fmoc synthesis on to the hydrogel of a Hi-Sense surface. After deprotection and washing, the cards were extensively washed in a sonication bath with a proprietary washing buffer.

### ELISA Screening

The binding of the antibodies to each of the synthesized peptides was tested by ELISA. The peptide arrays were incubated with primary antibody solution (overnight at 4° C.). After washing, the peptide arrays were incubated with a 1/1000 dilution of an antibody peroxidase conjugate (SBA, cat.nr.2010-05) for one hour at 25° C. After washing, the peroxidase substrate 2,2′-azino-di-3-ethylbenzthiazoline sulfonate (ABTS) and 2 μl/ml of 3% H₂O₂ were added. After one hour, the color development was measured. The color development was quantified with a charge coupled device (CCD)—camera and an image processing system.

### Design of Peptides

Chemically synthesized CLIPS peptides were synthesized (see also Methods section) according to the following designs.

### Set 1

### Mimic Type

Linear peptides

### Description

Linear 15mer peptides derived from human CD3ε residues 2-16. In each peptide one of the residues is replaced by all naturally occurring amino acids (except cysteine), creating a saturation mutagenesis library.
Sequences (first 10 shown)

AGNEEMGGITQTPYK
DGNEEMGGITQTPYK
GGNEEMGGITQTPYK
HGNEEMGGITQTPYK
LGNEEMGGITQTPYK
MGNEEMGGITQTPYK
NGNEEMGGITQTPYK
PGNEEMGGITQTPYK
QGNEEMGGITQTPYK
RGNEEMGGITQTPYK

### Set 2

Mimic Type: Linear peptides

### Description

Linear 15mer peptides derived from cynomolgus CD3ε residues 2-16. In each peptide one of the residues is replaced by all naturally occurring amino acids (except cysteine), creating a saturation mutagenesis library.

Sequences (first 10 shown)

AGNEEMGSITQTPYQ
DGNEEMGSITQTPYQ
GGNEEMGSITQTPYQ
HGNEEMGSITQTPYQ
LGNEEMGSITQTPYQ
MGNEEMGSITQTPYQ
NGNEEMGSITQTPYQ
PGNEEMGSITQTPYQ
QGNEEMGSITQTPYQ
RGNEEMGSITQTPYQ

### Set 3

Mimic Type: Linear peptides

### Description

Linear 15mer peptides derived from human CD3ε residues 5-20. In each peptide one of the residues is replaced by all naturally occurring amino acids (except cysteine), creating a saturation mutagenesis library.
Sequences (first 10 shown)

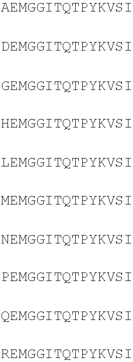

### Set 4

### Mimic Type

Linear peptides

### Description

Linear 15mer peptides derived from cynomolgus CD3ε residues 5-20. In each peptide one of the residues is replaced by all naturally occurring amino acids (except cysteine), creating a saturation mutagenesis library.
Sequences (first 10 shown)

AEMGSITQTPYQVSI
DEMGSITQTPYQVSI
GEMGSITQTPYQVSI
HEMGSITQTPYQVSI
LEMGSITQTPYQVSI
MEMGSITQTPYQVSI
NEMGSITQTPYQVSI
PEMGSITQTPYQVSI
QEMGSITQTPYQVSI
REMGSITQTPYQVSI

### Comparison of Samples

All five antibodies bind to linear peptides derived from the human and cynomolgus variant of the CD3ε N terminus in a very similar fashion, by absolutely requiring N4 (only to be supplanted by Histidine), and with a great preference for E6, for which limited substitutions are tolerated, however it seems that Glutamate is the most preferred residue at that position. None of the antibodies bound to peptides spanning residues 5-20. Within this group of five, three antibodies (Clone 2, Clone 3, and Clone 4) are more sensitive to mutations in the Cyno sequence than the other two (Clone 6, and Clone 10), in that the former group of three also is more sensitive to replacements of G3, E5, and/or G8. This observation is in line with the difference in affinity for the human and cynomolgus forms of the protein as determined by SPR (see Table 1).

### Conclusion

The analysis fine mapped the epitopes of the five antibodies, which bind the N terminus with an absolute requirement for N4 and E6, and share significant similarities. All antibodies bind both human and cynomolgus versions of CD3ε, despite the small differences in sequence adjacent to the core epitope.

### General Methods:

### Primary Sequence Analysis

The obtained sequence information of the corresponding heavy and light chain variable domains (VL and VH) was aligned and grouped according to sequence homology. The sets of rabbit variable domains were analyzed to identify unique clones and unique sets of CDRs. A combined alignment of the VL and VH domains was performed based on the joint amino acid sequences of both domains to identify unique clones. In addition to the alignment of the variable domains, the set of sequences of the six complementarity determining regions (CDRs) of each rabbit IgG clone were compared between different clones to identify unique sets of CDRs. These unique CDR sets were aligned using the multiple alignment tool COBALT and a phylogenetic tree was generated with the Neighbor Joining algorithm. The CDR sets were grouped based on sequence homology of the joined CDR sequences of each clone and a cluster threshold was determined based on sequence homology and identity. Based on the screening assay results and the cluster affiliation of the individual rabbit IgG clones candidates are selected for further analysis. Clones from different clusters were selected with the aim to proceed with high sequence diversity.

### Rabbit IgG Manufacturing

The rabbit IgG variable domains were cloned by RT-PCR amplification and ligation into a suitable mammalian expression vector for transient heterologous expression containing a leader sequence and the respective constant domains e.g. the pFUSE-rIgG vectors (Invivogen). The transient expression of the functional rIgG was performed by co-transfection of vectors encoding the heavy and light chains with the FreeStyle™ MAX system in CHO S cells. After cultivation for several days the supernatant of the antibody secreting cells was recovered for purification. Subsequently the secreted rabbit IgGs were affinity purified by magnetic Protein A beads (GE Healthcare). The IgG loaded beads were washed and the purified antibodies were eluted by a pH shift. The elution fractions were analyzed by sodium dodecyl sulfate polyacrylamide gel electrophoresis (SDS-PAGE), UV absorbance at 280 nm and size-exclusion high performance liquid chromatography (SE-HPLC) to ensure comparable quality of all samples.

### Engineering and Characterization of Humanized Single-Chain Fv Fragments and IgGs

The humanization of rabbit antibody clone comprised the transfer of the rabbit CDRs onto Numab's proprietary scFv acceptor framework of the Vκ1/VH3 type. In this process the amino acid sequence of the six CDR regions of a given rabbit clone was identified on the rabbit antibody donor sequence as described elsewhere (Borras, L. et al. 2010. JBC; 285:9054-9066) and grafted into the Numab acceptor scaffold sequence. In the case of rabbit clone clone-06, for example, the VL and VH sequences of the resulting humanized clone-06 are shown in SEQ ID NO: 21 and 22, respectively.

Humanized IgG constructs can be made in analogy to the method described in [00129].

SPR Assay for Determination of Binding Kinetics and Species Cross-Reactivity of Monoclonal anti-CD3 Antibodies

Binding affinities of monoclonal rabbit anti-CD3 antibodies were measured by surface plasmon resonance (SPR) using a MASS-1 SPR instrument (Sierra Sensors). For affinity measurements, an antibody specific for the Fc region of rabbit IgGs (Bethyl Laboratories, Cat. No. Al20-111A) was immobilized on a sensor chip (SPR-2 Affinity Sensor, Amine, Sierra Sensors) using a standard amine-coupling procedure. Rabbit monoclonal antibodies were captured by the immobilized anti-rabbit IgG antibody. Two-fold serial dilutions of human heterodimeric single-chain CD3εγ extracellular domain (produced in-house) ranging from 90 to 2.81 nM were injected into the flow cells for 3 min and dissociation of the protein from the IgG captured on the sensor chip was allowed to proceed for 5 min. After each injection cycle, surfaces were regenerated with two injections of 10 mM glycine-HCl. The apparent dissociation (kd) and association (ka) rate constants and the apparent dissociation equilibrium constant (KD) were calculated with the MASS-1 analysis software (Analyzer, Sierra Sensors) using one-to-one Langmuir binding model.

### Determination of Species Cross-Reactivity

Species cross-reactivity to cynomolgus monkey single-chain CD3εγ extracellular domain was measured using the same assay setup. Three-fold serial dilutions of cynomolgus monkey heterodimeric CD3εγ extracellular domain (produced in-house) ranging from 90 to 0.12 nM were injected into the flow cells for 3 min and dissociation of the protein from the IgG captured on the sensor chip was allowed to proceed for 5 min. After each injection cycle, surfaces were regenerated with two injections of 10 mM glycine-HCl. The apparent dissociation (kd) and association (ka) rate constants and the apparent dissociation equilibrium constant (KD) were calculated with the MASS-1 analysis software (Analyzer, Sierra Sensors) using one-to-one Langmuir binding model.

### Cell-based ELISA for Determination of Binding of Monoclonal Anti-CD3 Antibodies to CD3ε Expressed on the Cell Surface of T-Cells

Jurkat cells (clone E6-1), a human T cell line, were seeded at 300,000 cells/well in round bottom 96-well plates in 100 μl phosphate-buffered saline (PBS) containing 10% FBS. Five-fold serial dilutions of anti-CD3 rabbit monoclonal antibodies ranging from 90 nM to 0.0058 nM were added to the plates in 100 μl PBS containing 10% FBS. Binding of rabbit antibodies to CD3_{ε} expressed on the surface of Jurkat cells was detected by a secondary antibody specifically recognizing the Fc part of rabbit antibodies of the IgG subtype (JacksonImmuno Research, Cat. No. 111-035-046). This secondary antibody was linked to the enzyme horseradish peroxidase (HRP). HRP activity was measured by addition of TMB substrate (3,3′,5,5′-tetramethylbenzidine, KPL, Cat. No. 53-00-00), which in a colorimetric reaction is processed by the HRP. The color intensity of the processed substrate is directly proportional to the amount of anti-CD3 antibody bound to Jurkat cells. To quantify color intensity, light absorbance (optical density) at the respective wave length was measured using a microtiter plate reader (Infinity reader M200 Pro, Tecan).

To correct for unspecific binding of the antibodies to unknown components presented on the cell surface of Jurkat cells, a CD3εdeficient derivative of the Jurkat T cell line (J.RT3-T3.5) was used. Binding of the monoclonal antibodies to this cell line was measured as described above for the Jurkat cells. For quantification of specific binding to Jurkat cells, the optical density for binding to the negative control was subtracted from the optical density for binding to Jurkat cells. Data were analyzed using a four-parameter logistic curve fit using the Softmax Data Analysis Software (Molecular Devices), and the molar concentration of anti-CD3 antibody required to reach 50% binding (EC₅₀, mid-OD of the standard curve) was derived from dose response curves.

### Determination of Species Cross-Reactivity

Binding to cynomolgus monkey CD3 presented on the cell surface of HSC-F T cells was measured using the same assay setup. HSC-F cells, a cynomolgus monkey T cell line, were seeded at 300,000 cells/well in round bottom 96-well plates in 100 μl phosphate-buffered saline (PBS) containing 10% FBS. Five-fold serial dilutions of anti-CD3 rabbit monoclonal antibodies ranging from 18 nM to 0.0058 nM were added to the plates in 100 μl PBS containing 10% FBS. Binding of rabbit antibodies to cynomolgus monkey CD3_{ε} expressed on the surface of HSC-F cells was detected by a secondary antibody specifically recognizing the Fc part of rabbit antibodies of the IgG subtype (Jacksonlmmuno Research, Cat. No. 111-035-046). This secondary antibody was linked to the enzyme horseradish peroxidase (HRP). HRP activity was measured as described above.

To correct for unspecific binding of the antibodies to unknown components presented on the cell surface, a CD3_{ε} negative human B lymphoblast cell line (DB) was used. Binding of the monoclonal antibodies to this cell line was measured as described above. For quantification of specific binding to HSC-F cells, the optical density for binding to the negative control was subtracted from the optical density for binding to HSC-F cells. Data were analyzed using a four-parameter logistic curve fit using the Softmax Data Analysis Software (Molecular Devices), and the molar concentration of anti-CD3 antibody required to reach 50% binding (EC₅₀, mid-OD of the standard curve) was derived from dose response curves.

### T-Cell Activation by Monoclonal Anti-CD3 Antibodies: Induction of CD69 Expression

The potential of monoclonal rabbit anti-CD3 antibodies to induce T-cell activation was evaluated by measurement of induction of CD69 expression, an early T-cell activation marker, in Jurkat cells, described elsewhere (Gil et al, Cell.2002; 109: 901-912). For dose-response assays, Jurkat cells (100,000 cells/well) were stimulated for 24 h with 20 μg/ml, 5 μg/ml and 1.25 μg/ml of anti-CD3 antibodies. Prior to addition of anti-CD3 monoclonal antibodies to Jurkat cells, anti-CD3 antibodies were cross-linked by addition of 3-fold excess of a goat anti-rabbit IgG antibody (Bethyl Laboratories, Cat. No. A120-111A) and a rabbit anti-mouse IgG antibody (Jacksonlmmuno Research, Cat. No. 315-005-008) respectively when OKT3 (BioLegend, Cat. No. 317302) or TR66 (Novus Biologicals, Cat. No. NBP1-97446) were used. After stimulation, cells were stained for CD69 expression using a Phycoerithrin (PE)-labeled antibody specific for human CD69 (BioLegend, Cat. No. 310906) and then analyzed with a flow cytometer (FACS aria III, Becton Dickinson). As negative control unstimulated Jurkat cells incubated with the cross-linking antibody were stained with the anti-CD69 antibody described above. T-cell activation over time was assessed with a similar assay setup as described above. 100,000 Jurkat cells/well were stimulated for 0 h, 4 h, 15 h, 24 h, 48 h and 72 h with 5 μg/ml anti-CD3 antibodies that have been cross-linked as described above. Identical to the dose-response assay, CD69 expression was analyzed by flow cytometry.

Manufacturing of scDb Constructs

The nucleotide sequences encoding the various anti-IL5R×CDE3_{ε} scDb constructs were de novo synthesized and cloned into an adapted vector for E. coli expression that is based on a pET26b(+) backbone (Novagen). The expression construct was transformed into the E. coli strain BL12 (DE3) (Novagen) and the cells were cultivated in 2YT medium (Sambrook, J., et al., Molecular Cloning: A Laboratory Manual) as a starting culture. Expression cultures were inoculated and incubated in shake flasks at 37° C. and 200 rpm. Once an OD600 nm of 1 was reached protein expression was induced by the addition of IPTG at a final concentration of 0.5 mM. After overnight expression the cells were harvested by centrifugation at 4000 g. For the preparation of inclusion bodies the cell pellet was resuspended in IB Resuspension Buffer (50 mM Tris-HCl pH 7.5, 100 mM NaCl, 5 mM EDTA, 0.5% Triton X-100). The cell slurry was supplemented with 1 mM DTT, 0.1 mg/mL Lysozyme, 10 mM Leupeptin, 100 μM PMSF and 1 μM Pepstatin. Cells are lysed by 3 cycles of ultrasonic homogenization while being cooled on ice. Subsequently 0.01 mg/mL DNAse was added and the homogenate was incubated at room temperature for 20 min. The inclusion bodies were sedimented by centrifugation at 15000 g and 4° C. The IBs were resuspended in IB resuspension Buffer and homogenized by sonication before another centrifugation. In total a minimum of 3 washing steps with IB Resuspension Buffer were performed and subsequently 2 washes with IB Wash Buffer (50 mM Tris-HCl pH 7.5, 100 mM NaCl, 5 mM EDTA) were performed to yield the final IBs.

For protein refolding the isolated IBs were resuspended in Solubilization Buffer (100 mM Tris/HCl pH 8.0, 6 M Gdn-HCl, 2 mM EDTA) in a ratio of 5 mL per g of wet IBs. The solubilization was incubated for 30 min at room temperature until DTT was added at a final concentration of 20 mM and the incubation was continued for another 30 min. After the solubilization was completed the solution was cleared by 10 min centrifugation at 21500 g and 4° C. The refolding was performed by rapid dilution at a final protein concentration of 0.3 g/L of the solubilized protein in Refolding Buffer (typically: 100 mM Tris-HCl pH 8.0, 5.0 M Urea, 5 mM Cysteine, 1 mM Cystine). The refolding reaction was routinely incubated for a minimum of 14 h. The resulting protein solution was cleared by 10 min centrifugation at 8500 g and 4° C. The refolded protein was purified by affinity chromatography on Capto L resin (GE Healthcare). The isolated monomer fraction was analyzed by size-exclusion HPLC, SDS-PAGE for purity and UV/Vis spectroscopy for protein content. Buffer was exchanged into native buffer (50 mM Citrate-Phosphate pH 6.4, 200 mM NaCl) by dialysis.

SPR Assay for Determination of Binding Kinetics of Bispecific anti-CD3×IL5R scDbs

Binding affinities of anti-CD3×IL5R scDbs were measured by surface plasmon resonance (SPR) using a MASS-1 SPR instrument (Sierra Sensors). For affinity measurements to CD3, human heterodimeric single-chain CD3_{εγ} extracellular domain (produced in-house) is immobilized on a sensor chip (SPR-2 Affinity Sensor High Capacity, Amine, Sierra Sensors) using a standard amine-coupling procedure. Three-fold serial dilutions of scDbs ranging from 90 to 0.1 nM were injected into the flow cells for 3 min and dissociation of the protein from the CD3εγ immobilized on the sensor chip was allowed to proceed for 12 min. After each injection cycle, surfaces are regenerated with two injections of 10 mM Glycine-HCl (pH 2.0). For affinity measurements against IL5R, an antibody specific for the Fc region of human IgGs was immobilized on a sensor chip (SPR-2 Affinity Sensor High Capacity, Amine, Sierra Sensors) by amine-coupling. A human IL5R-Fc chimeric protein (Novus Biologicals) was captured by the immobilized antibody. Three-fold serial dilutions of scDbs specific for IL5R (90 nM −0.1 nM) are injected into the flow cells for three minutes and dissociation is monitored for 12 minutes. After each injection cycle, surfaces are regenerated with three injections of 10 mM Glycine-HCl (pH 1.5). The apparent dissociation (kd) and association (ka) rate constants and the apparent dissociation equilibrium constant (K_{D}) are calculated with the MASS-1 analysis software (Analyzer, Sierra Sensors) using one-to-one Langmuir binding model.

Binding of Bispecific Anti-CD3×IL5R scDbs to CD3ε Expressed on the Cell Surface of T-Cells and to IL5R Expressed on the Surface of CHO Cells (CHO-IL5R Cells)

Binding of scDbs to CD3ε expressed on the cell surface of Jurkat cells (clone E6-1, ATCC), a human T cell line, was analyzed by flow cytometry. To assess unspecific binding of the scDbs to unknown components presented on the cell surface of Jurkat cells a CD3ε deficient derivative of the Jurkat T cell line (J.RT3-T3.5, ATCC) was used. Binding of scDbs to IL5R expressed on the cell-surface was analyzed using transgenic CHO-IL5R cells (generated at ZHAW) and wild-type CHO cells (Invitrogen) were used as controls for unspecific binding. Both cell lines were incubated with 1 μg/mL and 10 μg/mL of scDbs for 1 hour and bound scDbs were detected by addition of RPE-labeled protein L (BioVision) and then analyzed with a flow cytometer (FACS aria III, Becton Dickinson). As negative control a scFv specific for an unrelated target was used. For the qualitative assessment of binding to Jurkat and CHO-IL5R cells the mean fluorescence intensity (MFI), reflecting the signal intensity at the geometric mean, was measured for both, the unspecific scFv as well as for the test scDbs. The difference of the MFI between test antibody and negative control antibody (ΔMFI) was calculated as a measure for binding. Furthermore, the normalized MFI was calculated by dividing the MFI of the test scDb through the MFI of the negative control scFv.

T-Cell Activation by Bispecific Anti-CD3×IL5R scDbs: Induction of IL-2 Secretion

The potential of anti-CD3×anti-IL5R scDbs to induce IL-2 expression in CD8+ cytotoxic T-cells in presence of target cells was evaluated as follows. Cytotoxic T-cells were freshly isolated from human blood by using the RosetteSep™ human CD8+ T-cell enrichment cocktail (STEMCELL Technologies) according to the manufacturer's instructions. CHO-IL5R cells (10,000 cells/well) were incubated with CD8+ cytotoxic T-cells at an effector:target ratio of 10:1 in presence of 10-fold serially diluted scDbs (100 nM to 0.001 nM) in 96 well microtiter plates. To assess unspecific stimulation of T-cells wild-type CHO cells were used as target cells. Supernatant was collected after 16 hours of co-incubation to measure IL-2 release. IL-2 release was quantified using a commercially available ELISA kit (BioLegend). Data were analyzed using a four-parameter logistic curve fit using the SoftMax® Pro data analysis Software (Molecular Devices), and the molar concentration of scDb required to induce half maximal IL-2 secretion (EC₅₀) is derived from dose-response curves.

scDb Mediated Lysis of IL5R Expressing CHO Cells by Cytotoxic T Cells

For assessment of the potential of bispecific anti-CD3×IL5R scDbs to induce target cell lysis a transgenic IL5R expressing CHO cell line was used (CHO-IL5R). Unstimulated human CD8+ T-cells isolated as described above were used as effector cells. Target cells were labeled with cell tox green dye (Promega) according to the manufacturer's instructions. Cell lysis was monitored by the CellTox™ green cytotoxicity assay (Promega). The assay measures changes in membrane integrity that occur as a result of cell death. The assay uses an asymmetric cyanine dye that is excluded from viable cells but preferentially stains the dead cell DNA. When the dye binds DNA in compromised cells, its fluorescence properties are substantially enhanced. Viable cells produce no appreciable increases in fluorescence. Therefore, the fluorescence signal produced by the binding interaction with dead cell DNA is proportional to cytotoxicity. Similarly as described above, labeled CHO-IL5R cells (10,000 cells/well) were incubated with CD8+ cytotoxic T-cells at an effector:target ratio of 10:1 in presence of 10-fold serially diluted scDbs (100 nM to 0.001 nM) in 96 well microtiter plates. To assess unspecific lysis of cells that do not express the target, T-cells were co-incubated with labeled wild-type CHO cells. Fluorescence intensity was analyzed after 88 h of incubation using a multi-mode microplate reader (FlexStation 3, Molecular Devices). Data were analyzed using a four-parameter logistic curve fit using the SoftMax® Pro data analysis Software (Molecular Devices), and the molar concentration of scDb required to induce half maximal target cell lysis (EC₅₀) was derived from dose-response curves.

TABLE 3
Residues most affecting binding of the different antibodies

| Clone ID NO. | SET 1 | SET 2 | SET 3 | SET 4 |
|---|---|---|---|---|
| clone-06 | N4; E6 | N4; E6 | binding low | binding low |
| clone-02 | N4; E6 | N4; E6; (G8) | binding low | binding low |
| clone-03 | N4; E6 | N4; E6; (G8) | binding low | binding low |
| clone-04 | N4; E6 | G3; E6 | binding low | binding low |
| clone-10 | N4; E6 | N4; E6 | binding low | binding low |

TABLE 4
Sequences of anti-CD3 antibodies

| | | Heavy | |
|---|---|---|---|
| | | Chain/ | |
| SEQ | Antibody | Light | |
| ID NO. | clone | Chain | Amino acid sequence |
| 1 | clone-01 | VL | AQVLTQTASSVSAAVGGTVTISCQSSESVY |
| | | | NNNRLSWFQQKPGQPPKQLIYSASSLASG |
| | | | VPSRFKGSGSGTQFTLTISDLECDDAATYY |
| | | | CQGEFSCSSADCFTFGGGTEVVVKGD |
| 2 | clone-01 | VH | QSVEESGGRLVTPGTPLTLTCTVSGFPLSS |
| | | | YAMIWVRQAPGKGLEWIGMILRAGNIYYAS |
| | | | WAKGRFTISKTSTTVDLKITSPTTEDTATYF |
| | | | CARRQYNTDGYPIGIGDLWGPGTLVTVSS |
| 3 | clone-02 | VL | AQVLTQTPSSVSAVVGGTVTISCQSSESVY |
| | | | SNNRLSWFQQKPGQPPKLLIYSASTLASGV |
| | | | PSRFKGSGSGTQFTLTITDLECDDAATYFC |
| | | | QGEFSCSSVDCFSFGGGTEVVVKGD |
| 4 | clone-02 | VH | QSLEESGGRLVTPGTPLTLTCTVSGFPLSA |
| | | | YAMIWVRQAPGKGLEWIGMIIRSGTVYYAN |
| | | | WAKGRFTISKTSTTVDLKITSPTTEDTATYF |
| | | | CARRHYNADGYPIGIGDLWGPGTLVTVSS |
| 5 | clone-03 | VL | AQVLTQTPSSVSAAVGGTVTISCQSNENIYS |
| | | | NNRLSWFQQKPGQPPNQLIYSASSLASGV |
| | | | PSRFKGSGSGTQFTLTISDLECDDAATYYC |
| | | | QGEFNCNSADCFTFGGGTEVVVKGD |
| 6 | clone-03 | VH | QSLEESGGRLVTPGTPLTLTCTVSGFPLNR |
| | | | YAMLWVRQAPGKGLEWIGLITRADKKYYAS |
| | | | WAKGRFTISKTSTTVDLEITGPTTEDTATYF |
| | | | CARRHYNTDGYPIAIGDLWGPGTLVTVSS |
| 7 | clone-04 | VL | AQVLTQTPSSVSAAVGGTVTINCQSSQSVY |
| | | | NNNRLSWFQQKPGQPPKLLIYTTSSLASGV |
| | | | PSRFKGSGSGTEFTLTISDLECADAATYYC |
| | | | QGEFSCSRADCFNFGGGTEVVVKGD |
| 8 | clone-04 | VH | QSLEESGGRLVKPDETLTLTCTVSGFPLSS |
| | | | YAMGWFRQAPGKGLEWIGMILRSDNTYYA |
| | | | SWAKGRFTISKTSTTVDLKITSPTTEDTATY |
| | | | FCARRHYNASGNPIAIGDLWGPGTLVTVSS |
| 9 | clone-06 | VL | AQVLTQTPSSVSAAVGGTVTISCQSSESVY |
| | | | NNKRLSWFQQKPGQPPKQLIYTASSLASGV |
| | | | PSRFKGSGSGTQFTLTISDLECDDAATYYC |
| | | | QGEFTCSNADCFTFGGGTEVVVKGD |
| 10 | clone-06 | VH | QSVEESGGRLVTPGTPLTLTCTVSGFPLSS |
| | | | YAMIWVRQAPGKGLEWIGMILRAGNIYYAS |
| | | | WVKGRVTISKTSTTVDLKITSPTTEDTATYF |
| | | | CARRHYNREGYPIGIGDLWGPGTLVTVSS |
| 11 | clone-09 | VL | AQVLTQTPSSVSAAVGGTVTISCQSNENIYS |
| | | | NNRLSWFQQKPGQPPNQLIYSASSLASGV |
| | | | PSRFKGSGSGTQFTLTISDLECDDAATYYC |
| | | | QGEFNCNSADCFTFGGGTEVVVKGD |
| 12 | clone-09 | VH | QSLEESGGRLVTPGTPLTLTCTVSGFPLNR |
| | | | YAMLWVRQAPGKGLEWIGLITRADKKYYAS |
| | | | WAKGRFTISKTSTTVDLEITGPTTEDTATYF |
| | | | CARRHYNTDGYPVAIGDLWGPGTLVTVSS |
| 13 | clone-10 | VL | AQVLTQTPSSVSAAVGGTATISCQSNENIYS |
| | | | NNRLSWFQQKAGQPPNQLIYSASSLASGV |
| | | | PSRFKGSGSGTQFTLTISDLECDDAATYYC |
| | | | QGEFSCSSADCFTFGGGTEVVVKGD |
| 14 | clone-10 | VH | QSLEESGGRLVTPGTPLTLTCTVSGFPLSS |
| | | | FAMLWVRQAPGKGLEWIGMIMRAHNMYYA |
| | | | SWAKGRFTISKTSTTVDLEITSPTTEDTATY |
| | | | FCARRHYNTYGYPIAIGDLWGPGTLVTVSS |
| 15 | clone-11 | VL | AQVLTQTPSSVSAAVGGTVTINCQSSQSVY |
| | | | NNNRLSWFQQKPGQPPKLLIYTASSLASGV |
| | | | PSRFKGSGSGTEFTLTISDLECADAATYYC |
| | | | QGEFSCSSADCFTFGGGTEVVVKGD |
| 16 | clone-11 | VH | QSLEESGGRLVTPGTPLTLTCTVSGFPLSS |
| | | | YAMGWFRQAPGKGLEWIGMILRADNTYYA |
| | | | SWVNGRFTISKTSTTVDLKITSPTTEDTATY |
| | | | FCARRHYNTYGYPVAIGDLWGPGTLVTVSS |
| 17 | clone-12 | VL | AQVLTQTPSSVSATVGGTVTISCQSNENIYS |
| | | | NNRLSWFQQKPGQPPKLLIYSASSLASGVP |
| | | | SRFKGSGSGTQFTLTISDLECDDAATYYCQ |
| | | | GEFNCNSADCFTFGGGTEVVVKGD |
| 18 | clone-12 | VH | QSLEESGGRLVTPGTPLTLTCTVSGFPLSR |
| | | | YAMLWVRQAPGKGLEWIGLITRADNKYYAS |
| | | | WAKGRFTISKTSTTVDLEITSPTTEDTATYF |
| | | | CARRHYNTDGYPIAIGDLWGPGTLVTVSS |
| 19 | consensus | VL | AQVLTQTX(P/A)SSVSAX(A/V/T)VGGTX(V/A) |
| | | | TIX(S/N)CQSX(S/N)X(E/Q)X(S/N)X(V/I)YX(S/ |
| | | | N)NX(N/K)RLSWFQQKX(P/A)GQPPX(K/N)X |
| | | | (Q/L)LIYX(S/T)X(A/T)SX(S/T)LASGVPSRFK |
| | | | GSGSGTX(Q/E)FTLTIX(S/T)DLECX(D/A)DA |
| | | | ATYX(Y/F)CQG EFX(S/N/T)CX(S/N)X(S/N/R) |
| | | | X(A/V)DCFX(T/S/N)FGGGTEVVVKGD |
| 20 | consensus | VH | QSX(L/V)EESGGRLVX(T/K)PX(G/D)X(T/E)X |
| | | | (P/T)LTLTCTVSGFPLX(S/N)X(S/A/R)X(Y/F)A |
| | | | MX(L/I/G)WX(V/F)RQAPGKGLEWIGX(M/L)I |
| | | | X(L/T/M/I)RX(A/S)X(D/G/H)X(N/K/T)X(K/T/I/M) |
| | | | YYAX(S/N)WX(A/V)X(K/N)GRX(F/V)TISKTS |
| | | | TTVDLX(K/E)ITX(S/G)PTTEDTATYFCARRX |
| | | | (H/Q)YNX(T/A/R)X(D/Y/S/E)GX(Y/N)PX(I/V)X |
| | | | (A/G)IGDLWGPGTLVTVSS |
| 21 | humanized | VL | DIQMTQSPSSLSASVGDRVTITCQSSESVY |
| | clone-06 | | NNKRLSWYQQKPGKAPKLLIYTASSLASGV |
| | | | PSRFSGSGSGTDFTLTISSLQPEDFATYYC |
| | | | QGEFTCSNADCFTFGQGTKLTVLG |
| 22 | humanized | VH | EVQLVESGGGLVQPGGSLRLSCAASGFPL |
| | clone-06 | | SSYAMIWVRQAPGKGLEWIGMILRAGNIYY |
| | | | ASWVKGRFTISRDNSKNTVYLQMNSLRAED |
| | | | TAVYYCARRHYNREGYPIGIGDLWGQGTLV |
| | | | TVSS |

[CDR1 to 3 shown in bold and underlined in SEQ ID NOs: 1 and 2 as representatives for all sequences]
[in SEQ ID NOs: 19 and 20: positions “X” are degenerate positions: respective degeneracy provided in square brackets behind individual “X”]

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

To the extent possible under the respective patent law, all patents, applications, publications, test methods, literature, and other materials cited herein are hereby incorporated by reference.

We claim:

1. An isolated antibody or functional fragment thereof comprising an antigen-binding region that is specific for an epitope of human CD3ε, wherein said epitope comprises amino acid residue N4 as residue that is critical for binding.

2. The isolated antibody or functional fragment thereof of claim 2, wherein said epitope further comprises amino acid residue E6 as residue that is involved in binding.

3. The isolated antibody or functional fragment thereof of claim 1, that is cross-reactive with cynomolgus CD3, particularly cynomolgus CD3ε, particularly having an affinity to cynomolgus monkey CD3ε that is less than 100-fold, particularly less than 30-fold, even more particularly less than 15-fold and most particularly less than 5-fold different to that of human CD3ε.

4. An isolated antibody or functional fragment thereof comprising an antigen-binding region that is specific for an epitope of human CD3, wherein said antibody or functional fragment thereof, is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M.

5. An isolated antibody or functional fragment thereof comprising an antigen-binding region that is specific for an epitope of human CD3, wherein said antibody or functional fragment thereof, when tested in an IgG format, upon cross-linking, is inducing T-cell activation at least 1.5-fold stronger than antibodies OKT-3 or TR66 after 24 h of stimulation at an IgG concentration of 1.25 μg/ml.

6. An isolated antibody or functional fragment thereof comprising an antigen-binding region that is specific for an epitope of human CD3, wherein said antibody or functional fragment thereof, when tested in an IgG format, upon cross-linking, is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml.

7. An isolated antibody or functional fragment thereof comprising an antigen-binding region that is specific for an epitope of human CD3, wherein said antibody or functional fragment thereof, when tested in an IgG format, upon cross-linking, is resulting in a dose-dependent homogeneous activation state of T-cells.

8. An isolated antibody or functional fragment thereof comprising an antigen-binding region that is specific for an epitope of human CD3, wherein said antibody or functional fragment thereof, when tested in an IgG format, (i) is binding to human CD3 with a dissociation constant for monovalent binding of less than 3.0×10⁻⁸ M, particularly less than 1.5×10⁻⁸ M, more particularly less than 1.2×10⁻⁸ M, and most particularly less than 1.0×10⁻⁸ M; and (iia), upon cross-linking, is inducing T-cell activation at least 1.5-fold stronger than antibodies OKT-3 or TR66 after 24 h of stimulation at an IgG concentration of 1.25 μg/ml; (iib) is resulting in T-cell activation, which lasts longer than with antibodies OKT-3 or TR66 as indicated by at least 1.5-fold greater increase in CD69 expression after 72 hours of stimulation at an IgG concentration of 1.25 μg/ml; (iic) is resulting in a dose-dependent homogeneous activation state of T-cells; and/or (iid) is specific for an epitope of human CD3ε, wherein said epitope comprises amino acid residue N4 as residue that is critical for binding.

9. The isolated antibody or functional fragment thereof of claim 4, wherein said epitope is located on the epsilon chain of human CD3.

10. The isolated antibody or functional fragment thereof of claim 1, wherein said binding to human CD3ε is determined by determining the affinity of said antibody or functional fragment thereof in an IgG format to the purified extracellular domain of heterodimeric CD3εγ of human origin using a surface plasmon resonance experiment, particularly using the following conditions: MASS-1 SPR instrument (Siena Sensors); capture antibody: antibody specific for the Fc region of said IgG immobilized on an SPR-2 Affinity Sensor chip, Amine, Siena Sensors, using a standard amine-coupling procedure; two-fold serial dilutions of human heterodimeric single-chain CD3εγ extracellular domain ranging from 90 to 2.81 nM, injection into the flow cells for 3 min and dissociation of the protein from the IgG captured on the sensor chip for 5 min, surface regeneration after each injection cycle with two injections of 10 mM glycine-HCl, calculation of the apparent dissociation (kd) and association (ka) rate constants and the apparent dissociation equilibrium constant (K_{D}) with the MASS-1 analysis software (Analyzer, Sierra Sensors) using one-to-one Langmuir binding model.

11. The isolated antibody or functional fragment thereof of claim 1, wherein said inducing of T-cell activation according to (iia) and/or (iic) is determined by determining the stimulation of CD69 expression by said isolated antibody or functional fragment thereof in an IgG format, particularly using the following conditions: stimulation of Jurkat cells (100,000 cells/well) for 24 h with 20 μg/ml, 5 μg/ml and 1.25 μg/ml of said isolated antibody or functional fragment thereof in an IgG format after prior cross-linking by addition of 3-fold excess of an anti-IgG antibody (control: OKT3 (BioLegend, Cat. No. 317302) or TR66 (Novus Biologicals, Cat. No. NBP1-97446), cross-linking with rabbit anti-mouse IgG antibody (JacksonImmuno Research, Cat. No. 315-005-008)); cell staining for CD69 expression after stimulation using a Phycoerithrin (PE)-labeled antibody specific for human CD69 (BioLegend, Cat. No. 310906), analysis with a flow cytometer (FACS aria III, Becton Dickinson); negative control: unstimulated Jurkat cells incubated with the cross-linking antibody stained with said anti-CD69 antibody.

12. The isolated antibody or functional fragment thereof of claim 1, wherein said longer lasting T-cell activation according to (iib) is determined by determining the time course of stimulation of CD69 expression by said isolated antibody or functional fragment thereof in an IgG format, particularly using the following conditions: stimulation of 100,000 Jurkat cells/well for 0 h, 4 h, 15 h, 24 h, 48 h and 72 h with 5 μg/ml of said isolated antibody or functional fragment thereof in an IgG format anti-CD3 antibodies that have been cross-linked as in claim 10 and analysis of CD69 expression by flow cytometry as in claim 10.

13. The isolated antibody or functional fragment thereof of claim 1, wherein said inducing of T-cell activation according to (iia) and/or (iic) is determined by determining the stimulation of IL-2 secretion by said isolated antibody or functional fragment thereof in an IgG format, particularly using the following conditions: stimulation of Jurkat cells (200,000 cells/well) with said isolated antibody or functional fragment thereof in an IgG format at a concentration of 5 μg/ml using 4 different assay setups: (a) stimulation of Jurkat cells with said isolated antibody or functional fragment thereof in an IgG format cross-linked by addition of 3-fold higher concentrations of an anti IgG antibody (control: OKT3 (BioLegend, Cat. No. 317302) or TR66 (Novus Biologicals, Cat. No. NBP1-97446), cross-linking with rabbit anti-mouse IgG antibody (Jacksonlmmuno Research, Cat. No. 315-005-008)); (b) T-cell activation in absence of cross-linking antibody; (c) immobilization of said cross-linking antibodies on the tissue culture plates by over-night incubation; (d) immobilization of said isolated antibody or functional fragment thereof in an IgG format (or of control antibodies) on the tissue culture plate by over-night incubation in absence of cross-linking antibodies; in each setup, one hour after addition, stimulation of cells with 10 ng/ml PMA and collection of supernatant after 24, 48 and 72 h to measure IL-2 release, quantified using a commercially available ELISA (BioLegend, Cat. No. 431801).

14. The isolated antibody or functional fragment thereof of claim 4, wherein said isolated antibody or functional fragment thereof is cross-reactive with cynomolgus CD3.

15. The isolated antibody or functional fragment thereof of claim 1, wherein said antibody or functional fragment thereof is (i) a rabbit antibody or functional fragment thereof, or (ii) an antibody or functional fragment thereof obtained by humanizing the rabbit antibody or functional fragment thereof of (i).

16. The isolated antibody or functional fragment thereof of claim 1, wherein said isolated antibody or functional fragment thereof comprises an antigen-binding region comprising a VH domain comprising a combination of one CDR1, one CDR2 and one CDR3 region present in SEQ ID NOs: 2, 4, 6, 8, 10, 12, 14, 16, 18, 20, and 22, particularly SEQ ID NOs: 4, 6, 10, and 22, more particularly SEQ ID NO: 10 and 22, particularly wherein said VH domain comprises framework domains selected from the framework domains present in SEQ ID NOs: 2, 4, 6, 8, 10, 12, 14, 16, 18, 20, and 22, particularly SEQ ID NOs: 4, 6, 10, and 22, more particularly SEQ ID NO: 10 and 22, and a VL domain comprising a combination of one CDR1, one CDR2 and one CDR3 region present in SEQ ID NOs: 1, 3, 5, 7, 9, 11, 13, 15, 17, 19, and 21, particularly SEQ ID NOs: 3, 5, 9, and 21, more particularly SEQ ID NO: 9 and 21, particularly wherein said VL domain comprises framework domains selected from the framework domains present in SEQ ID NOs: 1, 3, 5, 7, 9, 11, 13, 15, 17, 19, and 21, particularly SEQ ID NOs: 3, 5, 9, and 21, more particularly SEQ ID NO: 9 and 21.

17. The isolated antibody or functional fragment thereof of claim 16, wherein said isolated antibody or functional fragment thereof comprises an antigen-binding region comprising a VH domain comprising the combination of CDR1, CDR2 and CDR3 present in one of SEQ ID NOs: 2, 4, 6, 8, 10, 12, 14, 16, 18, 20, and 22, particularly SEQ ID NOs: 4, 6, 10, and 20, more particularly SEQ ID NO: 10 and 22, particularly wherein said VH domain comprises the combination of framework domains present in one of SEQ ID NOs: 2, 4, 6, 8, 10, 12, 14, 16, 18, and 20, and 22, particularly SEQ ID NOs: 4, 6, 10, and 22, more particularly SEQ ID NO: 10 and 22, and a VL domain comprising the combination of CDR1, CDR2 and CDR3 present in one of SEQ ID NOs: 1, 3, 5, 7, 9, 11, 13, 15, 17, 19, and 21, particularly SEQ ID NOs: 3, 5, 9, and 21, more particularly SEQ ID NO: 9 and 21, particularly wherein said VL domain comprises the combination of framework domains present in one of SEQ ID NOs: 1, 3, 5, 7, 9, 11, 13, 15, 17, 19, and 21, particularly SEQ ID NOs: 3, 5, 9, and 21, more particularly SEQ ID NO: 9 and 21.

18. The isolated antibody or functional fragment thereof of claim 17, wherein said isolated antibody or functional fragment thereof comprises an antigen-binding region comprising a VH domain selected from SEQ ID NOs: 2, 4, 6, 8, 10, 12, 14, 16, 18, 20, and 22, particularly SEQ ID NOs: 4, 6, 10, and 22, more particularly SEQ ID NO: 10 and 22, and a VL domain selected from SEQ ID NOs: 1, 3, 5, 7, 9, 11, 13, 15, 17, 19, and 21, particularly SEQ ID NOs: 3, 5, 9, and 21, more particularly SEQ ID NO: 9 and 21.

19. The isolated antibody or functional fragment thereof of claim 18, wherein said isolated antibody or functional fragment thereof comprises an antigen-binding region comprising a VH/VL domain combination selected from SEQ ID NO: 1/SEQ ID NO: 2; SEQ ID NO: 3/SEQ ID NO: 4; SEQ ID NO: 5/SEQ ID NO: 6; SEQ ID NO: 7/SEQ ID NO: 8, SEQ ID NO: 9/SEQ ID NO: 10, SEQ ID NO: 11/SEQ ID NO: 12, SEQ ID NO: 13/SEQ ID NO: 14, SEQ ID NO: 15/SEQ ID NO: 16, SEQ ID NO: 17/SEQ ID NO: 18, SEQ ID NO: 19/SEQ ID NO: 20, and SEQ ID NO: 21/SEQ ID NO: 22; particularly SEQ ID NO: 3/SEQ ID NO: 4; SEQ ID NO: 5/SEQ ID NO: 6; SEQ ID NO: 9/SEQ ID NO: 10, and SEQ ID NO: 21/SEQ ID NO: 22; more particularly SEQ ID NO: 9/SEQ ID NO: 10 and SEQ ID NO: 21/SEQ ID NO: 22.

20. An isolated antibody or functional fragment thereof binding to essentially the same epitope as the isolated antibody or functional fragment thereof of claim 16.

21. The isolated antibody or functional fragment thereof of claim 1, wherein said isolated antibody or functional fragment thereof comprises an antigen-binding region which is obtained by humanizing an antigen-binding region of claim 18.

22. A pharmaceutical composition comprising the isolated antibody or functional fragment thereof of claim 1, and optionally a pharmaceutically acceptable carrier and/or excipient.

23. A nucleic acid sequence or a collection of nucleic acid sequences encoding the isolated antibody or functional fragment thereof of claim 1.

24. A vector or a collection of vectors comprising the nucleic acid sequence or a collection of nucleic acid sequences of claim 23.

25. A host cell, particularly an expression host cell, comprising the nucleic acid sequence or the collection of nucleic acid sequences of claim 23, or a-vector or collection of vectors comprising said nucleic acid sequences or said collection of nucleic acid sequences.

26. A method for producing the isolated antibody or functional fragment thereof of claim 1, comprising the step of expressing the nucleic acid sequence or the collection of nucleic acid sequences encoding said isolated antibody or functional fragment thereof, or the vector or collection of vectors comprising said nucleic acid sequence or said collection of nucleic acid sequences, or the host cell, particularly an expression host cell comprising said nucleic acid sequences or said collection of nucleic acid sequences.
